(19) United States
(12) Patent Application Publication
(10) Pub. No.: US 2009/0239982 A1
(43) Pub. Date: Sep. 24, 2009

(54) Ultraviolet Absorbent and Heterocyclic Compound

(75) Inventors: Ichiro Amasaki, Odawara-shi (JP); Naoyuki Hanaki, Minami-ashigara-shi (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Minato-ku (JP)

(21) Appl. No.: 12/055,969

(22) Filed: Mar. 26, 2008

(30) Foreign Application Priority Data
Mar. 30, 2007 (JP) 2007-095446

(51) Int. Cl. C08K 5/3465; C07D 413/14; C08K 5/357; C07D 403/14

(52) U.S. Cl. 524/92; 544/73; 524/87; 544/284

(57) ABSTRACT

An ultraviolet absorbent represented by the following Formula (1) or (6):

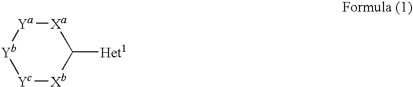

wherein, Het¹ represents a monovalent five- or six-membered aromatic heterocyclic residue; X^{a} and X^{b} each independently represent a heteroatom; Y^{a} to Y^{c} each independently represent a heteroatom or a carbon atom; the ring formed from carbon atom, X^{a}, X^{b}, Y^{a} to Y^{c} may have a double bond at any position and has at least one fused ring;

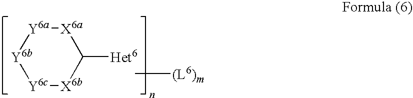

wherein, Het⁶ represents a monovalent five- or six-membered aromatic heterocyclic residue; X^{6a} and X^{6b} each independently represent a heteroatom; Y^{6a}, Y^{6b} and Y^{6c} each independently represent a heteroatom or a carbon atom; L⁶ represents a bi- to octa-valent connecting group; n is an integer of 2 or more, and m is an integer of 0 or more.

### FIELD OF THE INVENTION

The present invention relates to an ultraviolet absorbent and a heterocyclic compound.

### BACKGROUND OF THE INVENTION

An ultraviolet absorbent has been used together with various resins, for providing the resins with ultraviolet absorptivity. Inorganic or organic ultraviolet absorbents are used as the ultraviolet absorbents. Inorganic ultraviolet absorbents (see, for example, JP-A-5-339033 (“JP-A” means unexamined published Japanese patent application), JP-A-5-345639, and JP-A-6-56466 and others) are superior in durability such as weather resistance and heat resistance, but the degree of freedom in selecting the compound is limited, because the absorption wavelength is determined by the band gap of the compound, and in addition, there is no inorganic absorbent capable to absorb the light in a long-wavelength ultraviolet (UV-A) range of around 400 mm, and even if there is an absorbent capable to absorb the light in the long-wavelength ultraviolet light, the absorbent develops color, because it has an absorption also in the visible range.

In contrast, the degree of freedom in designing structures is much higher for organic ultraviolet absorbents, and thus, it is possible to obtain an absorbent having a various absorption wavelength by designing the absorbent structure properly.

Various organic ultraviolet absorbent systems have been studied, and two ways of thinking, namely, use of an absorbent having the maximum absorption wavelength in the long-wavelength ultraviolet range and use of a high concentration of absorbent are considered for absorbing the light in the long-wavelength ultraviolet range. However, the absorbents disclosed in JP-A-6-145387 and JP-A-2003-177235 and others having the maximum absorption wavelength in the long-wavelength ultraviolet range were lower in light stability, and their absorption capacity declines over time.

In contrast, benzophenone- and benzotriazole-based ultraviolet absorbents are relatively higher in light stability, and increase in concentration or film thickness leads to relatively clear blocking of the light in the longer-wavelength range (see, for example, JP-A-7-2005-517787 and JP-A-7-285927 and others). Further, benzoxazinone-based ultraviolet absorbents are also known (see, for example, JP-A-62-11744). However, when such an ultraviolet absorbent is coated as it is mixed with a resin and others, the film thickness is normally at most about dozens of μm. In order to block the light in the longer-wavelength range by this film thickness, it is necessary to add the ultraviolet absorbent in a significantly higher concentration. However, a mere increase in concentration only resulted in a problem of precipitation and bleed out of the ultraviolet absorbent during long-term use. There are some ultraviolet absorbents that are irritative to skin and accumulate in the body among the benzophenone- and benzotriazole-based ultraviolet absorbents, and thus, intensive care should have been given to these compounds during use.

### SUMMARY OF THE INVENTION

The present invention resides in an ultraviolet absorbent represented by the following Formula (1):

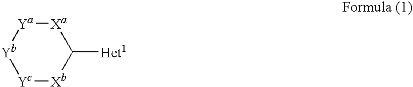

wherein, Het¹ represents a monovalent five- or six-membered aromatic heterocyclic residue; the aromatic heterocyclic residue may further be substituted;

X^{a} and X^{b} each independently represent a heteroatom, X^{a} and X^{b} may further be substituted;

Y^{a}, Y^{b} and Y^{c} each independently represent a heteroatom or a carbon atom; Y^{a}, Y^{b} and Y^{c} may further be substituted;

the ring formed from carbon atom, X^{a}, X^{b}, Y^{a}, Y^{b} and Y^{c} may have a double bond at any position;

the ring formed from carbon atom, X^{a}, X^{b}, Y^{a}, Y^{b} and Y^{c} has at least one fused ring.

Further, the present invention resides in an ultraviolet absorbent represented by the following Formula (6):

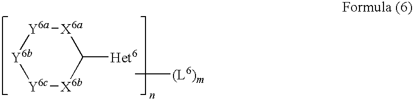

wherein, Het⁶ represents a monovalent five- or six-membered aromatic heterocyclic residue; the aromatic heterocyclic residue may further be substituted;

X^{6a} and X^{6b} each independently represent a heteroatom, X^{6a} and X^{6b} may further be substituted;

Y^{6a}, Y^{6b} and Y^{6c} each independently represent a heteroatom or a carbon atom;

Y^{6a}, Y^{6b} and Y^{6c} may further be substituted;

L⁶ represents a bi- to octa-valent connecting group;

n is an integer of 2 or more, and m is an integer of 0 or more.

Further, the present invention resides in a compound represented by the following Formula (6):

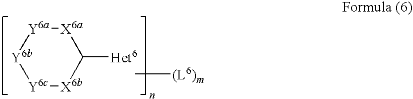

wherein, Het⁶ represents a monovalent five- or six-membered aromatic heterocyclic residue; the aromatic heterocyclic residue may further be substituted;

X^{6a} and X^{6b} each independently represent a heteroatom, X^{6a} and X^{6b} may further be substituted;

Y^{6a}, Y^{6b} and Y^{6c} each independently represent a heteroatom or a carbon atom;

Y^{6a}, Y^{6b} and Y^{6c} may further be substituted;

L⁶ represents a bi- to octa-valent connecting group;

n is an integer of 2 or more, and m is an integer of 0 or more.

Other and further features and advantages of the invention will appear more fully from the following description.

### DETAILED DESCRIPTION OF THE INVENTION

After studying intensively on heterocyclic compounds in detail, the inventors have found a compound that has a novel structure and is superior in light fastness and absorbs ultraviolet ray in the longer wavelength range that has been hitherto impossible to be covered, and thus, made the present invention.

The present invention provides the following means:

<1> An ultraviolet absorbent represented by the following Formula (1):

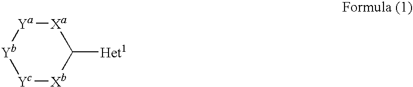

wherein, Het¹ represents a monovalent five- or six-membered aromatic heterocyclic residue; the aromatic heterocyclic residue may further be substituted;

X^{a} and X^{b} each independently represent a heteroatom, X^{a} and X^{b} may further be substituted;

Y^{a}, Y^{b} and Y^{c} each independently represent a heteroatom or a carbon atom; Y^{a}, Y^{b} and Y^{c} may further be substituted;

the ring formed from carbon atom, X^{a}, X^{b}, Y^{a}, Y^{b} and Y^{c} may have a double bond at any position;

the ring formed from carbon atom, X^{a}, X^{b}, Y^{a}, Y^{b} and Y^{c} has at least one fused ring.

<2> The ultraviolet absorbent described in <1>, wherein the ultraviolet absorbent represented by Formula (1) above is an ultraviolet absorbent represented by the following Formula (2):

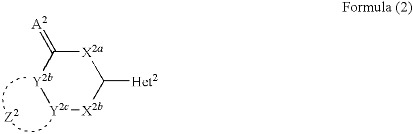

wherein, Het² is the same as Het¹ in Formula (1) above;

X^{2a} and X^{2b} each are the same as X^{a} and X^{b} in Formula (1) above;

Y^{2b} and Y^{2c} each are the same as Y^{b} and Y^{c} in Formula (1) above;

A² represents an oxygen atom or sulfur atom or ═NR^{a}, where R^{a} represents a hydrogen atom or a monovalent substituent group;

Z² represents an atom group needed for forming a four- to eight-membered ring together with Y^{2b} and Y^{2c}.

<3> The ultraviolet absorbent described in <2>, wherein the ultraviolet absorbent represented by Formula (2) above is an ultraviolet absorbent represented by the following Formula (3):

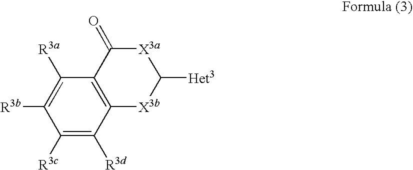

wherein, Het³ is the same as Het² in Formula (2) above;

X^{3a} and X^{3b} each are the same as X^{2a} and X^{2b} in Formula (2) above;

R^{3a}, R^{3b}, R^{3c} and R^{3d} each independently represent a hydrogen atom or a monovalent substituent group.

<4> The ultraviolet absorbent described in <3>, wherein the ultraviolet absorbent represented by Formula (3) above is an ultraviolet absorbent represented by the following Formula (4):

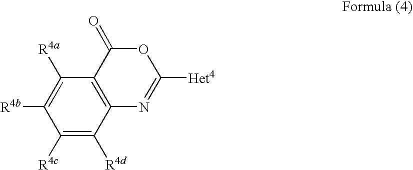

wherein, Het⁴ is the same as Het³ in Formula (3) above;

R^{4a}, R^{4b}, R^{4c} and R^{4d} are the same as R^{3a}, R^{3b}, R^{3c} and R^{3d} in Formula (3) above.

<5> The ultraviolet absorbent described in <4>, wherein the ultraviolet absorbent represented by Formula (4) above is an ultraviolet absorbent represented by the following Formula (5):

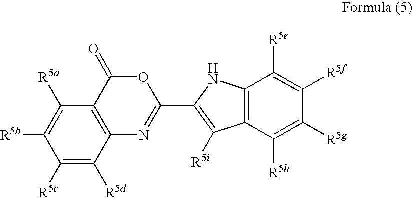

wherein, R^{5a}, R^{5b}, R^{5c} and R^{5d} are the same as R^{4a}, R^{4b}, R^{4c} and R^{4d} in Formula (4) above; R^{5e}, R^{5f}, R^{5g}, R^{5h} and R⁵ⁱ each independently represent a hydrogen atom or a monovalent substituent group.

<6> An ultraviolet absorbent represented by the following Formula (6):

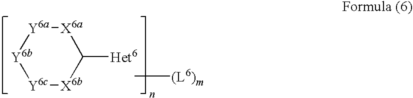

wherein, Het⁶ represents a monovalent five- or six-membered aromatic heterocyclic residue; the aromatic heterocyclic residue may further be substituted;

X^{6a} and X^{6b} each independently represent a heteroatom, X^{6a} and X^{6b} may further be substituted;

Y^{6a}, Y^{6b} and Y^{6c} each independently represent a heteroatom or a carbon atom;

Y^{6a}, Y^{6b} and Y^{6c} may further be substituted;

L⁶ represents a bi- to octa-valent connecting group;

n is an integer of 2 or more, and m is an integer of 0 or more.

<7> The ultraviolet absorbent described in <6>, wherein the ring formed from carbon atom, X^{6a}, X^{6b}, Y^{6a}, Y^{6b} and Y^{6c} has at least one fused ring.
<8> The ultraviolet absorbent described in <6> or <7>, wherein the ultraviolet absorbent represented by Formula (6) above is an ultraviolet absorbent represented by the following Formula (7):

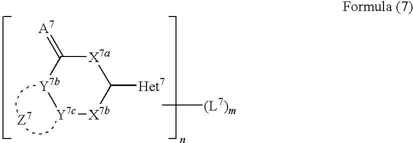

wherein, Het⁷ is the same as Het⁶ in Formula (6) above;

X^{7a} and X^{7b} each are the same as X^{6a} and X^{6b} in Formula (6) above;

Y^{7b} and Y^{7c} each are the same as Y^{6b} and Y^{6c} in Formula (6) above;

A⁷ represents an oxygen atom or sulfur atom or ═NR^{b} where R^{b} represents a hydrogen atom or a monovalent substituent group;

Z⁷ represents an atom group needed for forming a four- to eight-membered ring together with Y^{7b} and Y^{7c};

L⁷ is the same as L⁶ in Formula (6) above;

n and in each are the same as n and m in Formula (6) above.

<9> The ultraviolet absorbent described in <8>, wherein the ultraviolet absorbent represented by Formula (7) above is a compound represented by the following Formula (8):

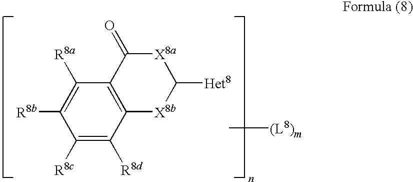

wherein, Het⁸ is the same as Het⁷ in Formula (7) above;

X^{8a} and X^{8b} each are the same as X^{7a} and X^{7b} in Formula (7) above;

R^{8a}, R^{8b}, R^{8c} and R^{8d} each independently represent a hydrogen atom or a monovalent substituent group;

L⁸ is the same as L⁷ in Formula (7) above;

n is an integer of 2 or more, and m is an integer of 0 or more.

<10> The ultraviolet absorbent described in <9>, wherein the compound represented by Formula (8) above is an ultraviolet absorbent represented by the following Formula (9):

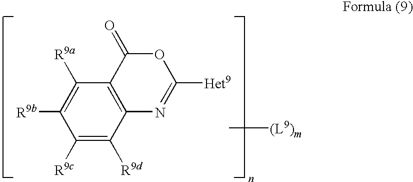

wherein, Het⁹ is the same as Het⁸ in Formula (8) above;

R^{9a}, R^{9b}, R^{9c} and R^{9d} are the same as R^{8a}, R^{8b}, R^{8c} and R^{8d} in Formula (8) above;

L⁹ is the same as L⁸ in Formula (8) above;

n is an integer of 2 or more, and m is an integer of 0 or more.

<11> The ultraviolet absorbent described in <10>, wherein the compound represented by Formula (9) above is an ultraviolet absorbent represented by the following Formula (10):

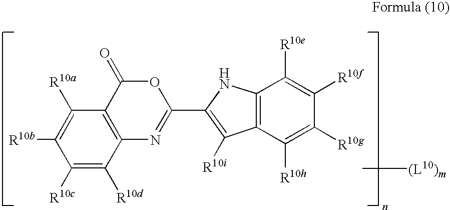

wherein, R^{10a}, R^{10b}, R^{10c} and R^{10d} are the same as R^{9a}, R^{9b}, R^{9c} and R^{9d} in Formula (9) above; R^{10e}, R^{10f}, R^{10g}, R^{10h} and R¹⁰ⁱ each independently represent a hydrogen atom or a monovalent substituent group;

L¹⁰ is the same as L⁹ in Formula (9) above;

n is an integer of 2 or more, and m is an integer of 0 or more.

<12> A polymer material, comprising a polymer substance and the ultraviolet absorbent described in any one of <1> to <11>.
<13> A compound represented by Formula (6) above.
<14> The compound described in <13>, wherein the compound is produced by using the compound represented by Formula (1) above.

Hereinafter, the present invention will be described in detail.

First, the ultraviolet absorbent represented by Formula (1) above will be described.

In Formula (1) above, Het¹ represents a monovalent five- or six-membered aromatic heterocyclic residue having at least one hetero atom. Het¹ may be a fused ring.

Examples of the hetero atoms include boron, nitrogen, oxygen, silicon, phosphorus, sulfur, selenium, tellurium, and the like, preferably, nitrogen, oxygen and sulfur atoms, more preferably nitrogen and sulfur atoms, and particularly preferably a nitrogen atom. If the ring has two or more hetero atoms, the hetero atoms may be the same as or different from each other.

Examples of the aromatic heterocycle prepared by adding one hydrogen atom to a monovalent aromatic heterocyclic residue include pyrrole, pyrazole, imidazole, 1,2,3-triazole, 1,2,4-triazole, pyridine, pyridazine, pyrimidine, pyrazine, 1,3,5-triazine, furan, thiophene, oxazole, isoxazole, thiazole, isothiazole, 1,2,3-oxadiazole, 1,3,4-thiadiazole, indole, benzofuran, benzothiophene, indazole, benzisoxazole, benzoisothiazole, benzimidazole, benzoxazole, benzothiazole, quinoline, isoquinoline, chinolin, quinazoline, phthalazine, quinoxaline, acridine, phenanthridine, pteridine, carbazole, β-carboline, purine, coumarin, dibenzofuran, phenothiazole, 1,10-phenanthroline, flavone and the like. The aromatic heterocycle is preferably thiophene, pyrrole, benzothiophene, benzofuran, or indole, more preferably thiophene or indole, and particularly preferably indole. The binding position of the aromatic heterocycles is arbitrary. For example, in the case of thiophene, a five-membered heterocyclic compound, the 2- and 3-sites are exemplified as its binding position. Alternatively, in the case of pyridine, a six-membered heterocyclic compound, the 2-, 3- and 4-sites are exemplified as its binding position.

The aromatic heterocyclic residue may have a substituent group(s). The substituent group is, for example, a monovalent substituent group. Examples of the monovalent substituent groups (hereinafter, referred to as R) include halogen atoms (e.g., fluorine atom, chlorine atom, bromine atom, and iodine atom), alkyl groups having 1 to 20 carbon atoms (e.g., methyl and ethyl), aryl groups having 6 to 20 carbon atoms (e.g., phenyl and naphthyl), a cyano group, a carboxyl group, alkoxycarbonyl groups (e.g., methoxycarbonyl), aryloxycarbonyl groups (e.g., phenoxycarbonyl), substituted or unsubstituted carbamoyl groups (e.g., carbamoyl, N-pheylcarbamoyl and N,N-dimethylcarbamoyl), alkylcarbonyl groups (e.g., acetyl), arylcarbonyl groups (e.g., benzoyl), a nitro group, substituted or unsubstituted amino groups (e.g., amino, dimethylamino and anilino), acylamino groups (e.g., acetamido and ethoxycarbonylamino), sulfonamido groups (e.g., methane sulfonamide), imido groups (e.g., succinimido and phthalimido), imino groups (e.g., benzylideneamino), a hydroxy group, alkoxy groups having 1 to 20 carbon atoms (e.g., methoxy), aryloxy groups (e.g., phenoxy), acyloxy groups (e.g., acetoxy), alkylsulfonyloxy groups (e.g., methanesulfonyloxy), arylsulfonyloxy groups (e.g., benzenesulfonyloxy), a sulfo group, substituted or unsubstituted sulfamoyl groups (e.g., sulfamoyl and N-phenylsulfamoyl), alkylthio groups (e.g., methylthio), arylthio groups (e.g., phenylthio), alkylsulfonyl groups (e.g., methanesulfonyl), arylsulfonyl groups (e.g., benzenesulfonyl), heterocyclic groups having 6 to 20 carbon atoms (e.g., pyridyl, morpholino), and the like. The substituent group may be further substituted, and the multiple substituent groups, if present, may be the same as or different from each other. The substituent groups then are, for example, the monovalent substituents R described above. The substituent groups may bind together to form a ring.

The substituent group is preferably an alkyl group, an alkoxy group, or an aryl group, more preferably an alkyl or aryl group, and particularly preferably an alkyl group.

X^{a} and X^{b} each independently represent a hetero atom. Examples of the hetero atoms include boron, nitrogen, oxygen, silicon, phosphorus, sulfur, selenium, tellurium, and the like, preferably, nitrogen, oxygen and sulfur atoms, more preferably nitrogen and oxygen atoms. Further, X^{a} and X^{b} may have a substituent group(s). The substituent groups then are, for example, the monovalent substituents R described above.

Y^{a}, Y^{b} and Y^{c} each independently represent a heteroatom or a carbon atom. The atoms constituting Y^{a}, Y^{b} and Y^{c} include, for example, carbon atom, nitrogen atom, oxygen atom, sulfur atom and the like. The atoms constituting Y^{a}, Y^{b} and Y^{c} are preferably carbon atom, nitrogen atom, and oxygen atom, more preferably carbon atom and nitrogen atom, still more preferably carbon atom, and particularly preferably all carbon atoms. The atom may further be substituted, and the substituent groups may bind together to form a ring, which may additionally be fused with another ring. The substituent groups then are, for example, the monovalent substituents R described above.

The ring formed from carbon atom, X^{a}, X^{b}, Y^{a}, Y^{b} and Y^{c} (ring bound to the aromatic heterocyclic residue represented by Het¹ above) may have a double bond at any position. However, the ring has at least one fused ring.

Hereinafter, specific examples of the rings formed from carbon atom, X^{a}, X^{b}, Y^{a}, Y^{b} and Y^{c} (the ring bound to the aromatic heterocyclic residue represented by Het¹ above) will be listed, but the present invention is not limited thereby. The wavy lines in the specific example indicate the binding site to the aromatic heterocyclic residue represented by Het¹ above.

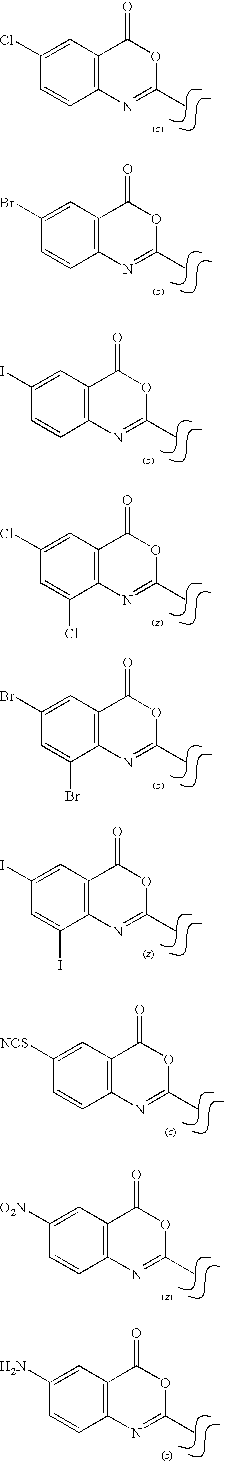

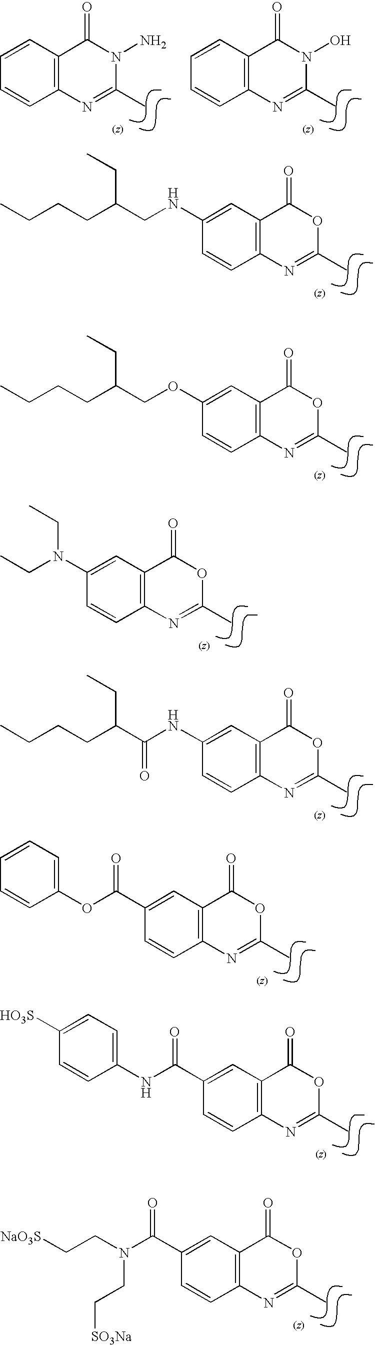

The ultraviolet absorbent represented by Formula (1) above is preferably an ultraviolet absorbent represented by Formula (2) above. Hereinafter, the ultraviolet absorbent represented by Formula (2) above will be described.

Het² is the same as Het¹ in Formula (1) above and the favorable examples thereof are also the same.

X^{2a} and X^{2b} each are the same as X^{a} and X^{b} in Formula (1) above and the favorable examples thereof are also the same. X^{2a} and X^{2b} may be different from each other, and at least one of them more preferably represents a nitrogen atom, and particularly preferably, X^{2a} represents an oxygen atom and X^{2b} a nitrogen atom.

Y^{2b} and Y^{2c} each are the same as Y^{b} and Y^{c} in Formula (1) above and the favorable examples thereof are also the same.

A² represents an oxygen atom or sulfur atom or ═NR^{a} (R^{a} represents a hydrogen atom or a monovalent substituent group. The substituent groups then are, for example, the monovalent substituents R described above.). Preferably, A² is an oxygen atom or ═NR^{a}, more preferably an oxygen atom.

Z² represents an atom group needed for forming a four- to eight-membered ring together with Y^{2b} and Y^{2c}. This ring may have a substituent group(s), which may further have a fused ring. Examples of the rings formed include aliphatic hydrocarbon rings such as cyclohexane and cyclopentane; aromatic hydrocarbon rings such as benzene and naphthalene ring; and heterocycles such as pyridine, pyrrole, pyridazine, thiophene, imidazole, furan, pyrazole, oxazole, triazole, thiazole, or the benzo-fused rings thereof, and the like. Preferable are aromatic hydrocarbon rings and heterocycles. More preferable are aromatic hydrocarbon rings, and particularly preferable is a benzene ring.

Further, the ultraviolet absorbent represented by Formula (2) is preferably an ultraviolet absorbent represented by Formula (3) above. Hereinafter, the ultraviolet absorbent represented by Formula (3) above will be described.

Het³ is the same as Het² in Formula (2) above and the favorable examples thereof are also the same.

X^{3a} and X^{3b} each are the same as X^{2a} and X^{2b} in Formula (2) above and the favorable examples thereof are also the same.

R^{3a}, R^{3b}, R^{3c} and R^{3d} each independently represent a hydrogen atom or a monovalent substituent group. The substituent groups then are, for example, the monovalent substituents R described above. Any two substituent groups among R^{3a} to R^{3d} may bind together to form a ring, which may have additionally a fused ring. R^{3a}, R^{3b}, R^{3c} and R^{3d} each are preferably a hydrogen atom, an alkyl group having 10 or less carbon atoms, an alkoxy group having 10 or less carbon atoms, or a hydroxy group, more preferably a hydrogen atom or an alkoxy group having 10 or less carbon atoms, still more preferably a hydrogen atom, and particularly preferably, R^{3a} to R^{3d} are all hydrogen atoms.

Further, the ultraviolet absorbent represented by Formula (3) is preferably an ultraviolet absorbent represented by Formula (4) above. Hereinafter, the ultraviolet absorbent represented by the following Formula (4) will be described.

Het⁴ is the same as Het³ in Formula (3) above and the favorable examples thereof are also the same.

R^{4a}, R^{4b}, R^{4c} and R^{4d} each are the same as R^{3a}, R^{3b}, R^{3c} and R^{3d} in Formula (3) above and the favorable examples thereof are also the same.

Further, the ultraviolet absorbent represented by Formula (4) is preferably an ultraviolet absorbent represented by Formula (5) above. Hereinafter, the ultraviolet absorbent represented by the following Formula (5) will be described.

R^{5a}, R^{5b}, R^{5c} and R^{5d} each are the same as R^{4a}, R^{4b}, R^{4c} and R^{4d} in Formula (4) above and the favorable examples thereof are also the same.

R^{5e}, R^{5f}, R^{59g}, R^{5h} and R⁵ⁱ each independently represent a hydrogen atom or a monovalent substituent group. The substituent groups then are, for example, the monovalent substituents R described above. Any two substituent groups among R^{5e} to R⁵ⁱ may bind together to form a ring, which may have additionally a fused ring. R^{5e}, R^{5f}, R^{5g}, R^{5h} and R⁵ⁱ each preferably represent a hydrogen atom, an alkyl group having 10 or less carbon atoms, an alkoxy group having 10 or less carbon atoms, or a hydroxy group, more preferably a hydrogen atom or an alkoxy group having 10 or less carbon atoms, still more preferably a hydrogen atom, and particularly preferably, R^{5e} to R⁵ⁱ are all hydrogen atoms.

The compound represented by any one of Formulae (1) to (5) may be prepared by any method. Examples of the methods include those disclosed in known patent documents and non-patent documents, specifically those described in the Examples of JP-A-51-10086, p. 7, left column, lines 21 to 36; “Bioorganic & Medicinal Chemistry Letters”, 2001, vol. 11, p. 1801-1804; “Molecules”, 2002, vol. 7, p. 353-362, and others. For example, exemplary compound (M-1) can be prepared in reaction of 2-thiophenecarbonyl chloride with anthlianilic acid. Further, exemplary compound (M-10) can be prepared in reaction of 2-pyridylcarbonyl chloride with anthranilic acid. Alternatively, exemplary compound (M-28) can be prepared in reaction of 2-quinolinecarboxylic acid chloride with anthlianilic acid. Yet alternatively, exemplary compound (M-44) can be prepared in reaction of 2-indolecarbonyl chloride with 5-hydroxyanthranilic acid. Yet alternatively, exemplary compound (M-52) can be prepared in reaction of the exemplary compound (M-1) with 2-naphthoyl chloride. Yet alternatively, exemplary compound (M-66) can be prepared in reaction of 2-thiophenecarbonyl chloride with 3,5-dichloroanthranilic acid. Yet alternatively, exemplary compound (M-72) can be prepared in reaction of the exemplary compound (M-1) with hydroxylamine. Yet alternatively, exemplary compound (M-74) can be prepared in reaction of the exemplary compound (M-24) with 2-ethylhexyl bromide.

Hereinafter, specific examples of the compounds represented by any one of Formulae (1) to (5) will be described below, but the present invention is not restricted thereby.

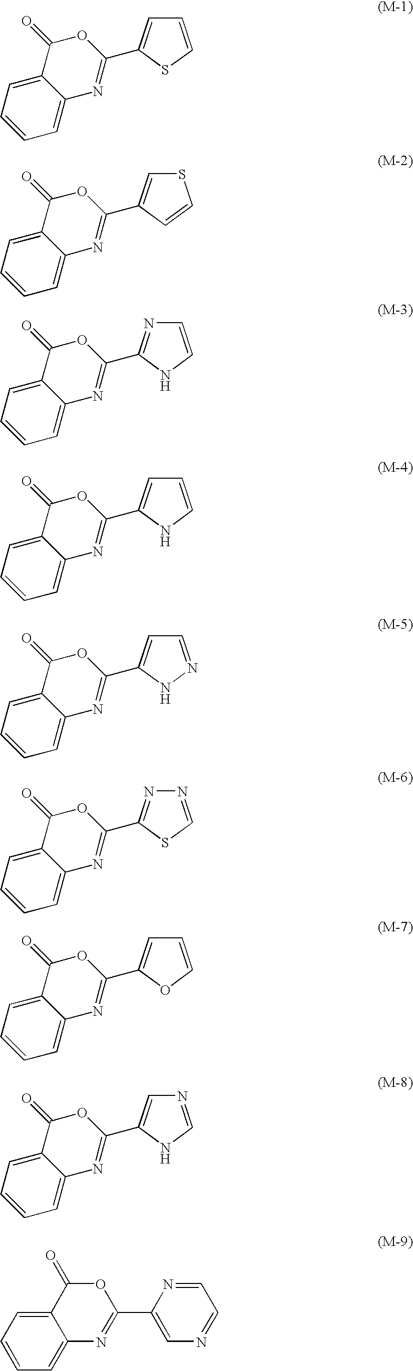

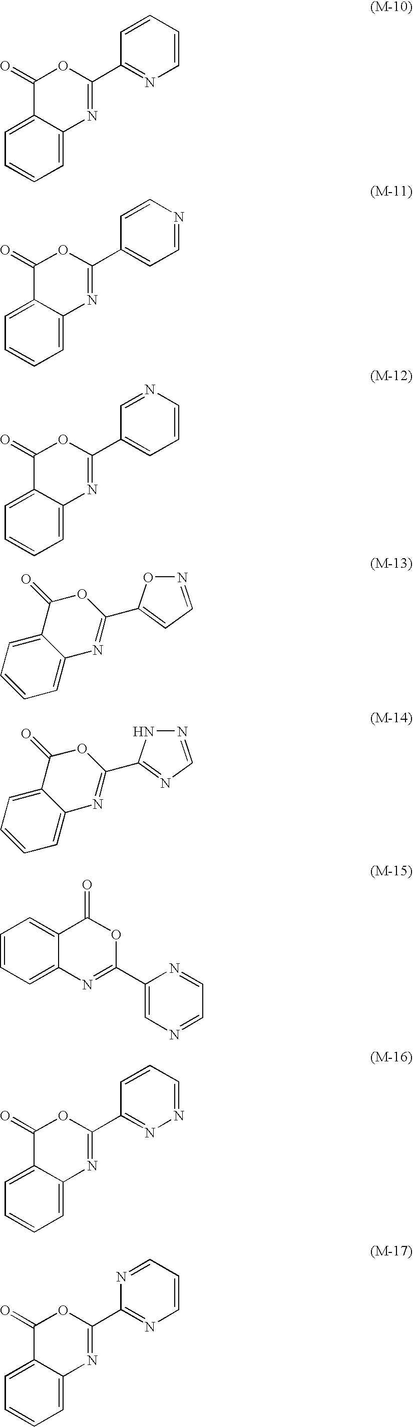

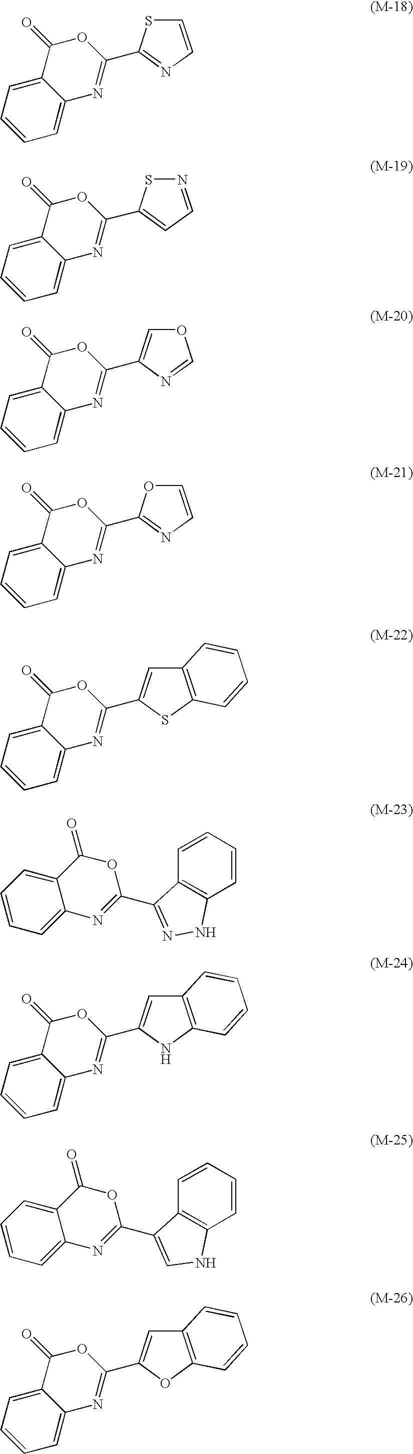

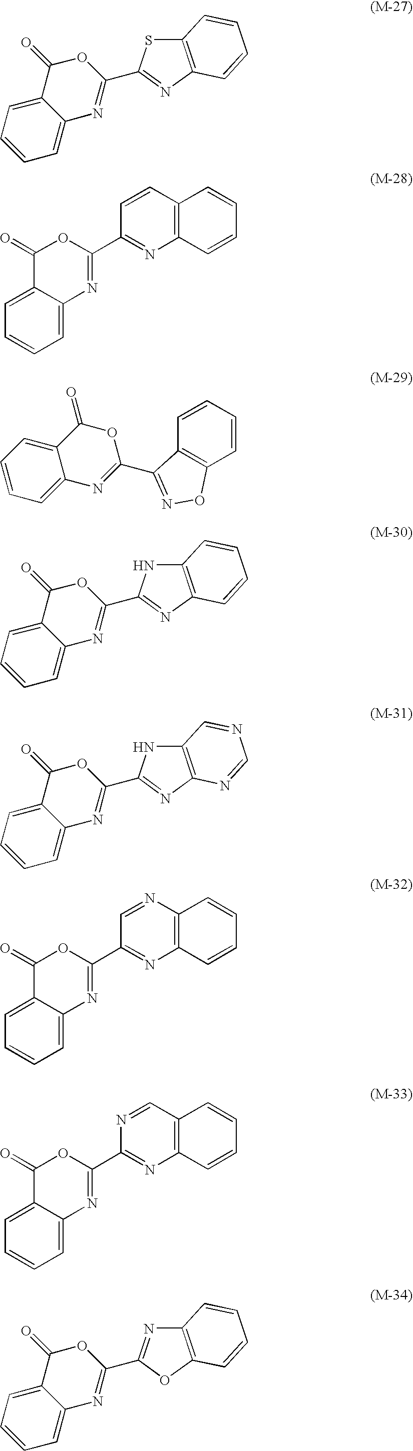

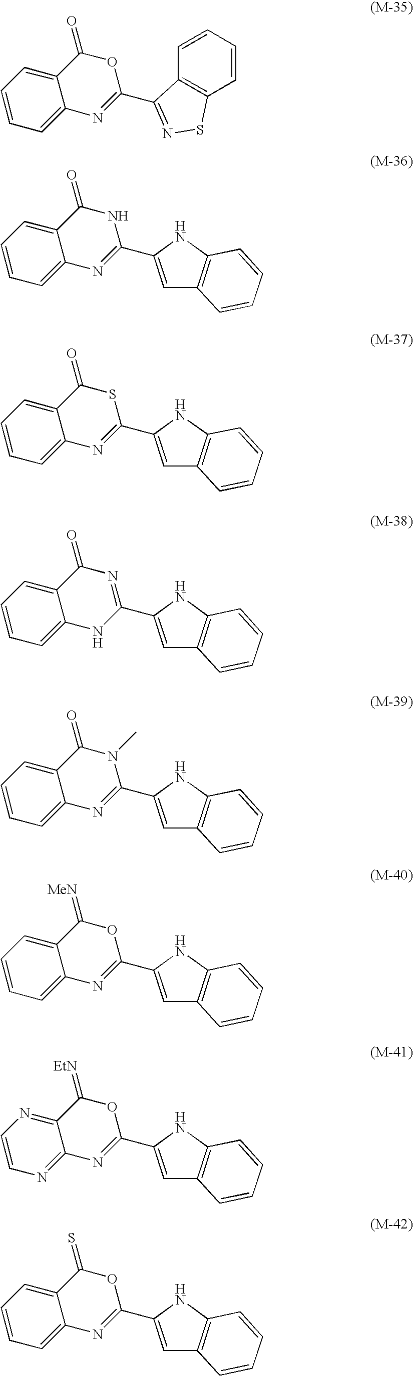

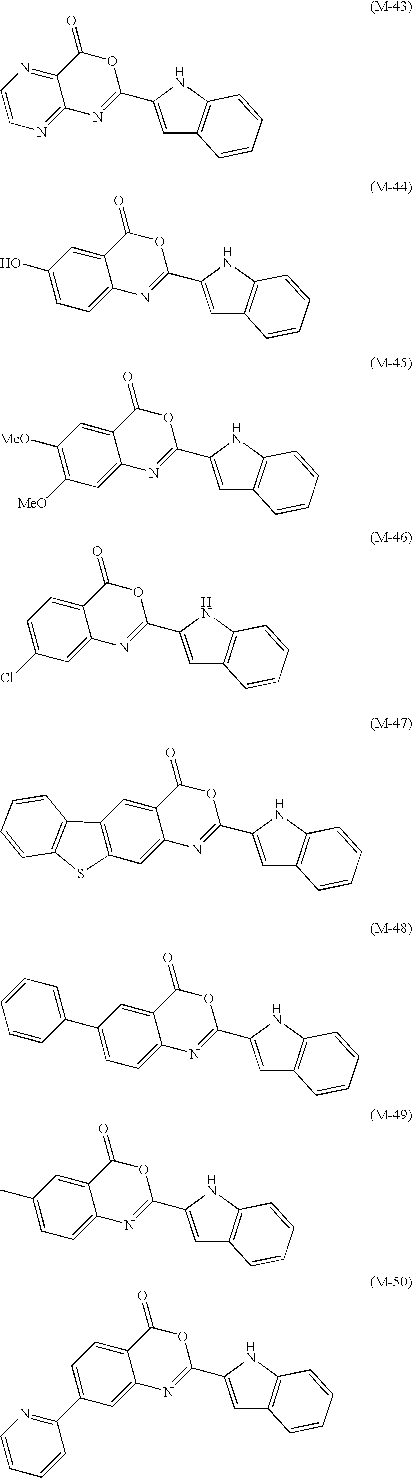

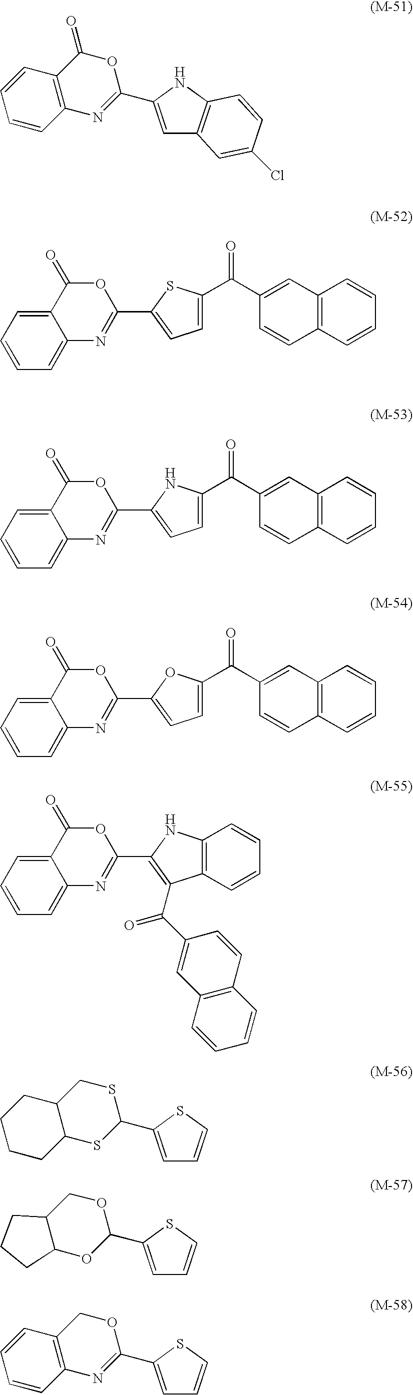

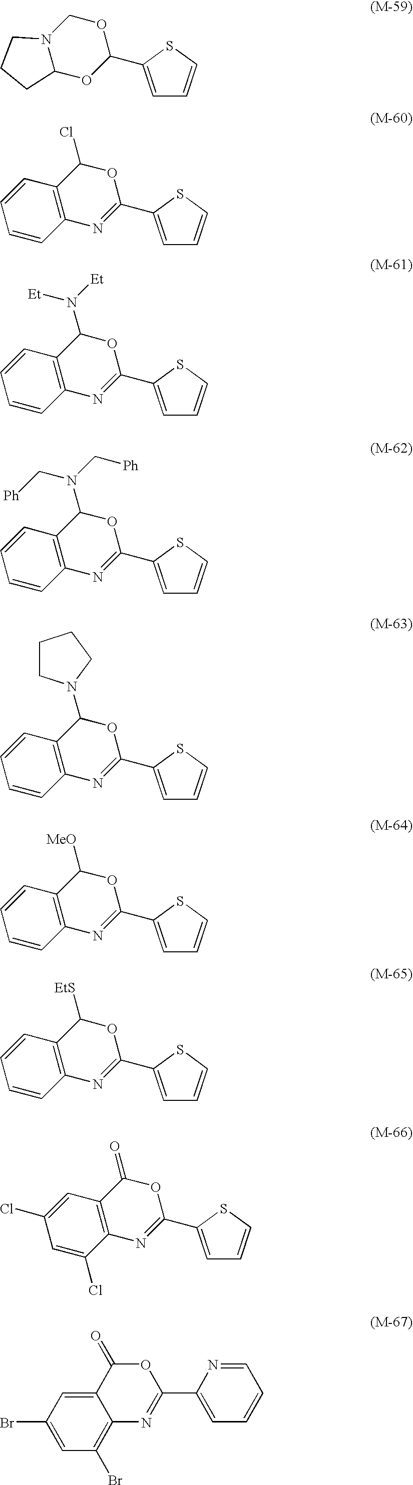

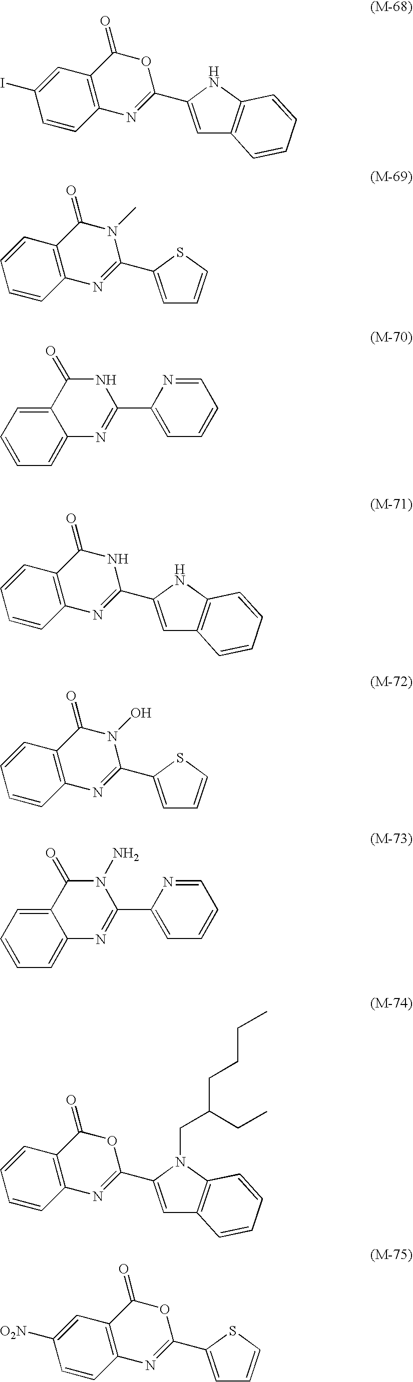

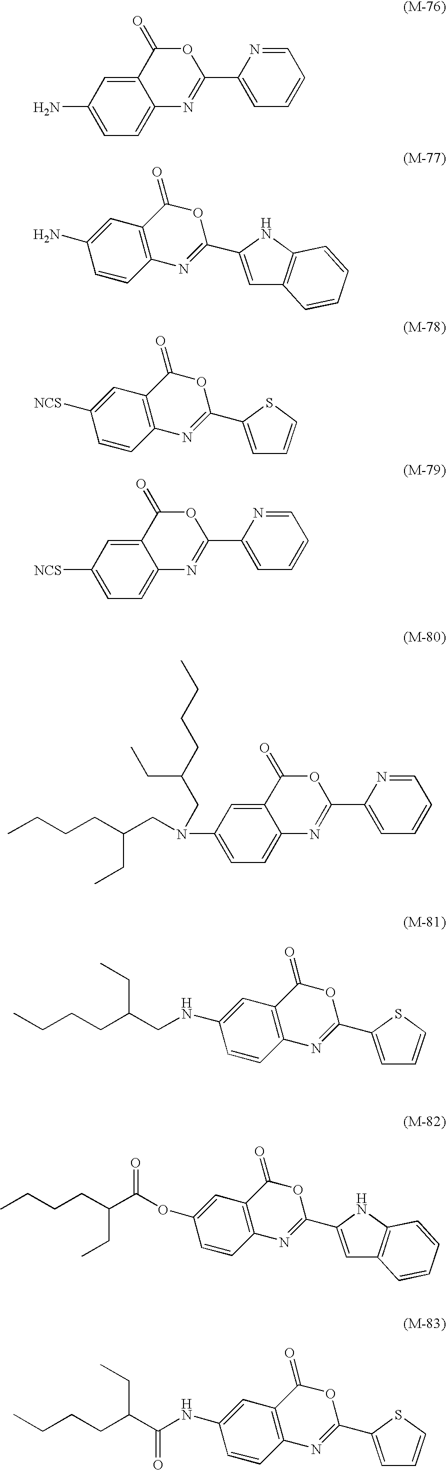

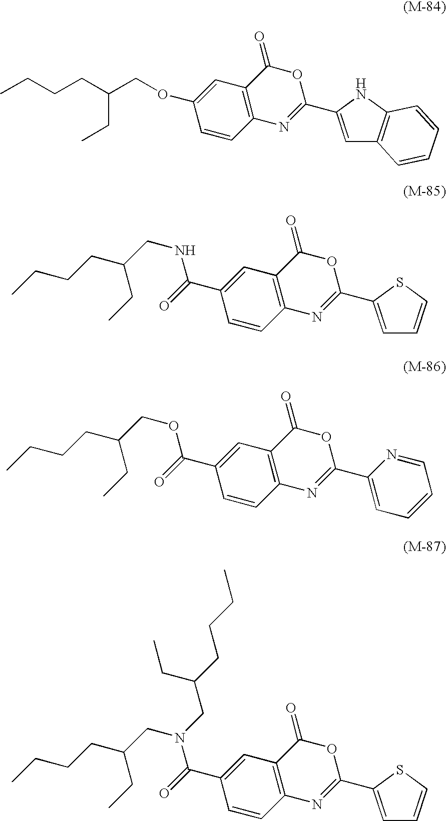

Next, the ultraviolet absorbent represented by Formula (6) above will be described.

The compound represented by Formula (6) above has a structure wherein multiple structures represented by Formula (1) above bind to each other directly or via a connecting group L.

Hereinafter, the ultraviolet absorbent represented by Formula (6) above will be described.

In formula (6) above, Het⁶ represents a monovalent five- or six-membered aromatic heterocyclic residue having at least one hetero atom. Het⁶ is the same as Het¹ in Formula (1) above and the favorable examples thereof are also the same.

X^{6a} and X^{6b} each independently represent a heteroatom. X^{6a} and X^{6b} each are the same as X^{a} and X^{b} in Formula (1) above and the favorable examples thereof are also the same.

Y^{6a}, Y^{6b} and Y^{6c} each independently represent a heteroatom or a carbon atom Y^{6a}, Y^{6b} and Y^{6c} each are the same as Y^{a}, Y^{b} and Y^{c} in Formula (1) above and the favorable examples thereof are also the same.

L⁶ represents a t-valent connecting group. The valency t is the number of the residues in L⁶ where hydrogen atoms and substituent groups are removed from the structure represented by Formula (1) above at any position, and L⁶ is preferably a bi- to octa-valent connecting group, preferably a bi- to quadri-valent connecting group. The connecting group is a single bond, a t-valent aliphatic hydrocarbon group, an aromatic hydrocarbon group, or an atom or an atom group containing an atom selected from hetero atoms such as nitrogen atom, sulfur atom and oxygen atom. Preferably, L⁶ is a bivalent connecting group. Examples of the bivalent connecting groups include connecting groups having 1 to 20 carbon atoms containing one or more of the following groups: alkylene groups (such as methylene, ethylene, propylene, butylene, pentylene and octylene), arylene groups (such as phenylene and naphthylene), alkenylene groups (such as ethenylene and propenylene), alkynylene groups (such as ethynylene and propynylene), amido groups, ester groups, sulfonamido group, sulfonic ester groups, ureido groups, sulfonyl groups, sulfinyl groups, thioether groups, ether groups, a carbonyl group, amino groups, and heterocyclic bivalent groups (such as 6-chloro-1,3,5-triazine-2,4-diyl group, pyrimidine-2,4-diyl group, and quinoxaline-2,3-diyl group). These substituent groups may have a ring (aromatic, non-aromatic hydrocarbon ring, or heterocycle). The connecting group above may have additionally the monovalent substituent R described above.

L⁶ may bond to a residue obtained by removing hydrogen atoms and substituent groups from the structure represented by Formula (1) at any position. In Formula (6) above, L⁶ preferably bonds to Het⁶.

Among the specific examples of L⁶, the ethylene, butylene and octylene groups respectively have the following structures. The wavy lines in the specific examples indicate the binding site to a residue obtained by eliminating a hydrogen atom or a substituent group from the structure represented by Formula (1) at any position.

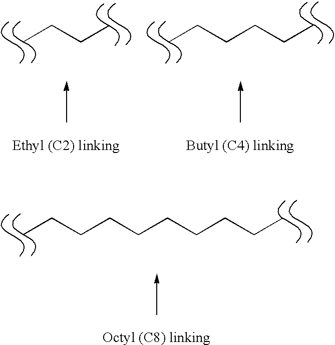

n is an integer of 2 or more, and m is an integer of 0 or more. n is preferably 2 or 3, and m is preferably 1 or 2.

In Formula (6), n residues in the structure represented by Formula (1) may be the same as or different from each other.

Specific examples of the ring A, the aromatic heterocyclic residue Het and the connecting group L are shown in the following Tables 1 to 15. The ring formed from X^{a}, X^{b}, Y^{a} to Y^{c} and carbon atom in Formula (1) and the ring formed from X^{6a}, X^{6b}, Y^{6a} to Y^{6c} and carbon atom in Formula (6) above are designated as A, the aromatic heterocyclic residue represented by Het¹ in Formula (1) above and the aromatic heterocyclic residue represented by Het⁶ in Formula (6) above are designated as Het, and the connecting group represented by L⁶ above is designated as L.

TABLE 1

| A | Het | L |
|---|---|---|
| 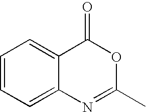 | 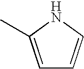 | 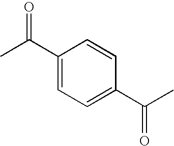 |
| 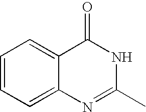 | 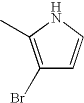 | 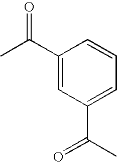 |
| 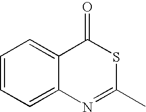 | 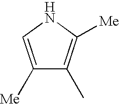 | 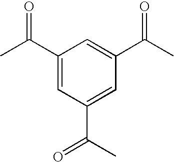 |
| 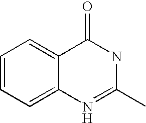 | 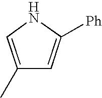 | 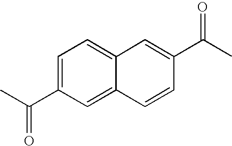 |
| 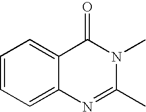 | 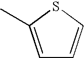 | 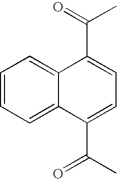 |
| 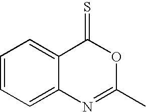 | 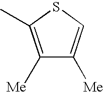 | 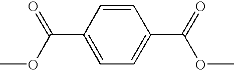 |

TABLE 2

| A | Het | L |
|---|---|---|
| 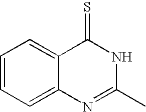 | 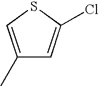 | 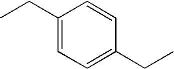 |
| 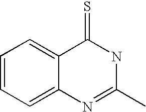 | 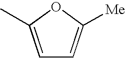 | 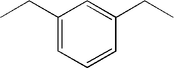 |
| 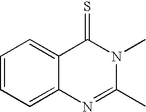 | 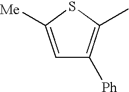 | 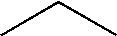 |
| 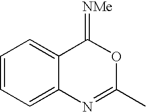 | 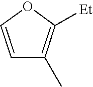 | 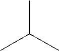 |
| 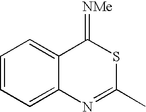 | 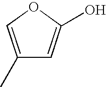 | 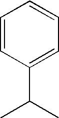 |
| 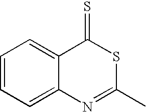 | 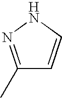 | 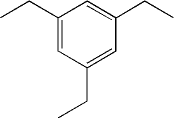 |
| 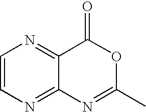 | 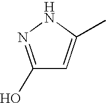 | 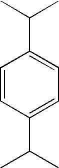 |
| 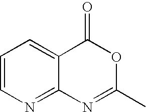 | 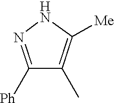 | 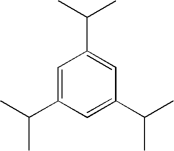 |

TABLE 3

| A | Het | L |
|---|---|---|
| 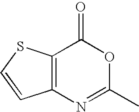 | 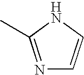 | 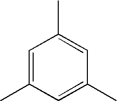 |
| 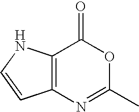 | 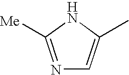 | 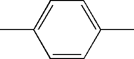 |
| 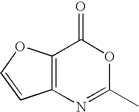 | 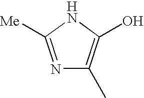 | 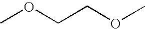 |
| 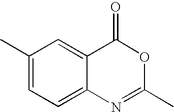 | 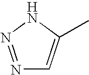 | 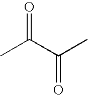 |
| 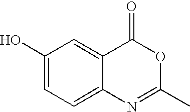 | 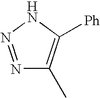 | |
| 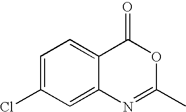 | 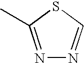 | |
| 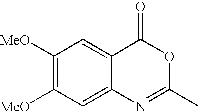 | 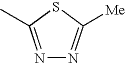 | |
| 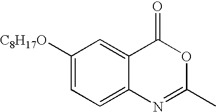 | 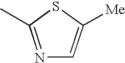 | |
| 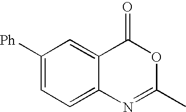 | 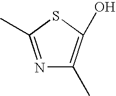 | |

TABLE 4

| A | Het | L |
|---|---|---|
| 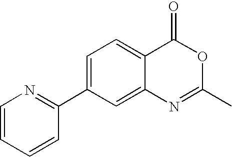 | 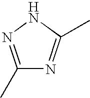 | |
| 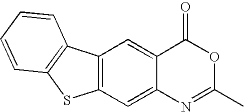 | 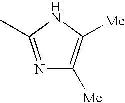 | |
| 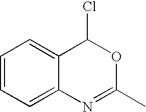 | 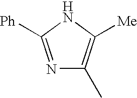 | |
| 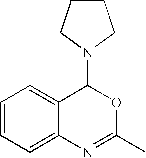 | 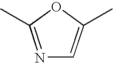 | |
| 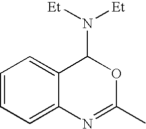 | 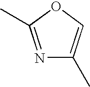 | |
| 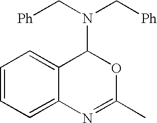 | 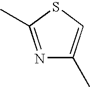 | |
| 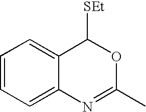 | 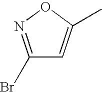 | |

TABLE 5

| A | Het | L |
|---|---|---|
| 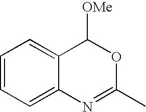 | 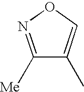 | |
| 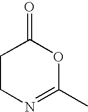 | 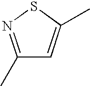 | |
| 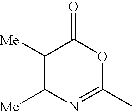 | 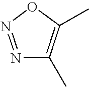 | |
| 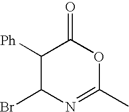 | 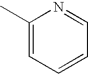 | |
| 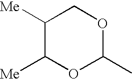 | 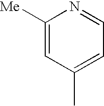 | |
| 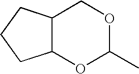 | 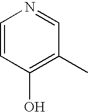 | |
| 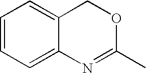 | 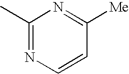 | |
| 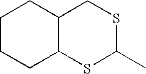 | 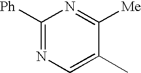 | |

TABLE 6

| A | Het | L |
|---|---|---|
| 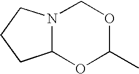 | 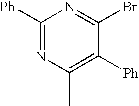 | |
| 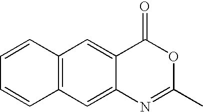 | 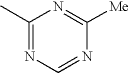 | |
| 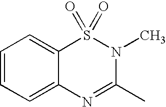 | 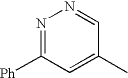 | |
| 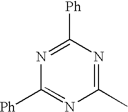 | 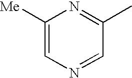 | |
| 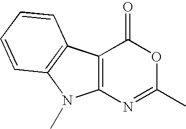 | 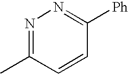 | |
| 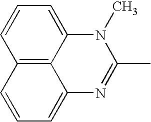 | 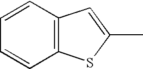 | |
| | 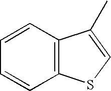 | |
| | 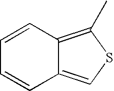 | |
| | 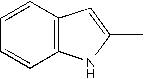 | |

TABLE 7

| A | Het | L |
|---|---|---|
| | 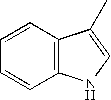 | |
| | 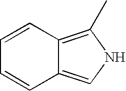 | |
| | 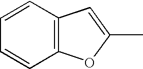 | |
| | 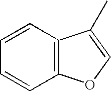 | |
| | 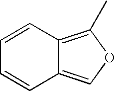 | |
| | 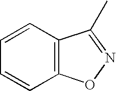 | |
| | 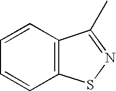 | |
| | 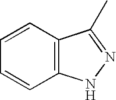 | |
| | 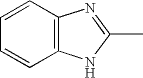 | |

TABLE 8

| A | Het | L |
|---|---|---|
| | 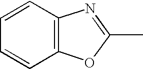 | |
| | 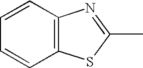 | |
| | 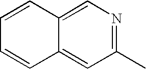 | |
| | 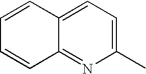 | |
| | 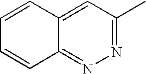 | |
| | 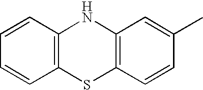 | |
| | 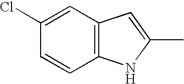 | |
| | 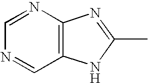 | |
| | 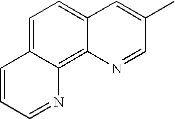 | |

TABLE 9

| A | Het | L |
|---|---|---|
| | 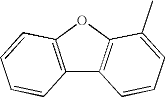 | |
| | 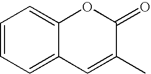 | |
| | 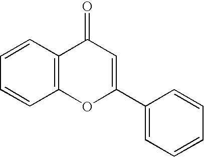 | |
| | 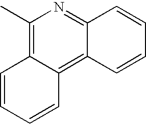 | |
| | 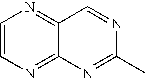 | |
| | 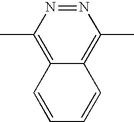 | |
| | 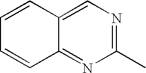 | |

TABLE 10

| A | Het | L |
|---|---|---|
| | 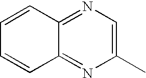 | |
| | 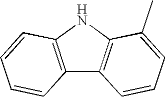 | |
| | 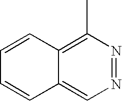 | |
| | 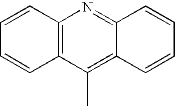 | |
| | 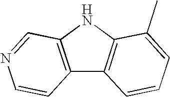 | |
| | 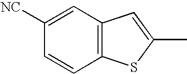 | |
| | 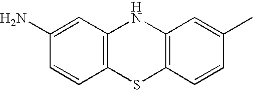 | |
| | 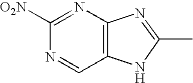 | |

TABLE 11

| A | Het | L |
|---|---|---|
| | 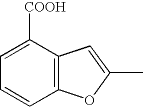 | |
| | 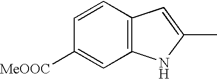 | |
| | 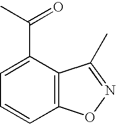 | |
| | 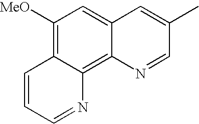 | |
| | 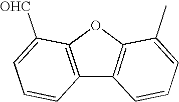 | |
| | 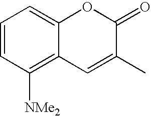 | |
| | 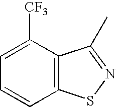 | |

TABLE 12

| A | Het | L |
|---|---|---|
| | 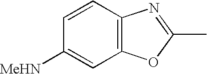 | |
| | 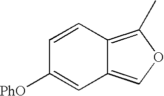 | |
| | 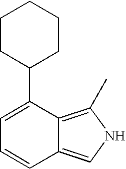 | |
| | 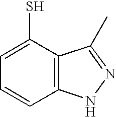 | |
| | 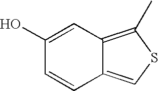 | |
| | 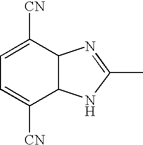 | |
| | 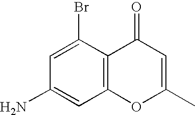 | |

TABLE 13

| A | Het | L |
|---|---|---|
| | 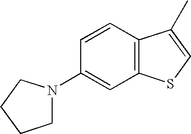 | |
| | 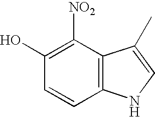 | |
| | 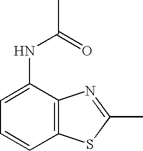 | |
| | 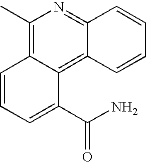 | |
| | 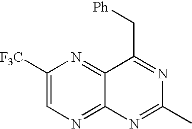 | |
| | 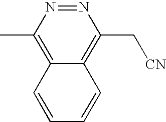 | |

TABLE 14

| A | Het | L |
|---|---|---|
| | 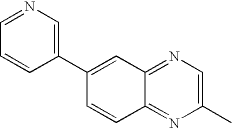 | |
| | 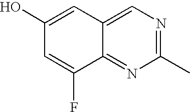 | |
| | 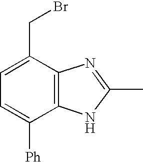 | |
| | 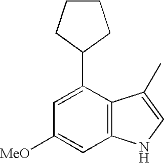 | |
| | 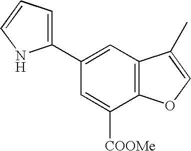 | |
| | 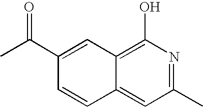 | |

TABLE 15

| A | Het | L |
|---|---|---|
| | 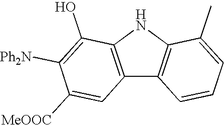 | |
| | 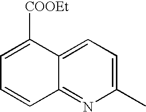 | |
| | 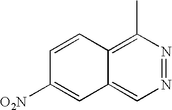 | |
| | 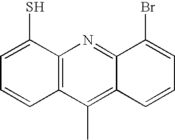 | |
| | 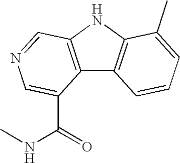 | |
| | 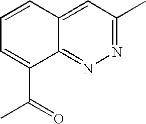 | |

Further, the compound represented by Formula (6) above is preferably a compound represented by Formula (7) above. Hereinafter, the compound represented by Formula (7) above will be described.

Het⁷ is the same as Het⁶ in Formula (6) above and the favorable examples thereof are also the same.

X^{7a} and X^{7b} each are the same as X^{6a} and X^{6b} in Formula (6) above and the favorable examples thereof are also the same. X^{7a} and X^{7b} may be the same as or different from each other, and at least one of them more preferably represents a nitrogen atom, and particularly preferably, X^{7a} represents an oxygen atom and X^{7b} a nitrogen atom.

Y^{7b} and Y^{7c} each are the same as Y^{6b} and Y^{6c} in Formula (6) above and the favorable examples thereof are also the same.

A⁷ represents an oxygen atom or sulfur atom or ═NR (R represents a hydrogen atom or a monovalent substituent group. The substituent groups then are, for example, the monovalent substituents R described above.). A⁷ is the same as A² in Formula (2) above and the favorable examples thereof are also the same.

Z⁷ represents an atom group needed for forming a four- to eight-membered ring together with Y^{7b} and Y^{7c}. Z⁷ is the same as Z² in Formula (2) above and the favorable examples thereof are also the same.

n and m each are the same as n and m in Formula (6) above and the favorable examples thereof are also the same.

Further, the compound represented by Formula (7) above is preferably a compound represented by Formula (8) above. Hereinafter, the compound represented by Formula (8) above will be described.

Het⁸ is the same as Het⁷ in Formula (7) above and the favorable examples thereof are also the same.

X^{8a} and X^{8b} each are the same as X^{7a} and X^{7b} in Formula (7) above and the favorable examples thereof are also the same.

R^{8a}, R^{8b}, R^{8c} and R^{8d} each independently represent a hydrogen atom or a monovalent substituent group. R^{8a}, R^{8b}, R^{8c} and R^{8d} each are the same as R^{3a}, R^{3b}, R^{3c} and R^{3d} in Formula (3) above and the favorable examples thereof are also the same.

L⁸ is the same as L⁷ in Formula (7) above and the favorable examples thereof are also the same.

n and m each are the same as n and m in Formula (7) above and the favorable examples thereof are also the same.

Further, the compound represented by Formula (8) above is preferably a compound represented by Formula (9) above. Hereinafter, the compound represented by Formula (9) above will be described.

Het⁹ is the same as Het⁸ in Formula (8) above and the favorable examples thereof are also the same

R^{9a}, R^{9b}, R^{9c} and R^{9d} each are the same as R^{8a}, R^{8b}, R^{8c} and R^{8d} in Formula (8) above and the favorable examples thereof are also the same.

L⁹ is the same as L⁸ in Formula (8) above and the favorable examples thereof are also the same.

n and m each are the same as n and m in Formula (8) above and the favorable examples thereof are also the same.

Further, the compound represented by Formula (9) above is preferably a compound represented by Formula (10) above. Hereinafter, the compound represented by Formula (10) above will be described.

R^{10a}, R^{10b}, R^{10c} and R^{10d} each are the same as R^{9a}, R^{9b}, R^{9c} and R^{9d} in Formula (9) above and the favorable examples thereof are also the same.

R^{10e}, R^{10f}, R^{10g}, R^{10h} and R¹⁰ⁱ each independently represent a hydrogen atom or a monovalent substituent group. R^{10e} to R¹⁰ⁱ each are the same as R^{5a} to R⁵ⁱ in Formula (5) above and the favorable examples thereof are also the same.

L¹⁰ is the same as L⁹ in Formula (9) above and the favorable examples thereof are also the same.

n and m each are the same as n and m in Formula (9) above and the favorable examples thereof are also the same.

The compound represented by any one of Formulae (6) to (10) may be prepared by any method. Examples thereof include a method of producing it by constructing the structure represented by Formula (1) above by using a raw material having a partial structure represented by Formula (1) above that is previously bonded directly to or via L⁶, a method of producing it by connecting the compound represented by Formula (1) above directly to or via L⁶ as a raw material, and the like. Considering availability of raw materials and difficulty in obtaining a desired compound without control of the reaction at multiple reaction points, the method of producing it by connecting the compound represented by Formula (1) above compound as a raw material is easier and thus more preferable. When the method is used, the compound can be prepared according to the methods described in Preparative Examples for the compounds represented by Formula (1) or the methods described in following known literatures: “Chemistry Letters”, 2004, vol. 33, p. 288-289; “Tetrahedron Letters”, 2006, vol. 47, p. 3019-3022; “Tetrahedron Letters”, 1995, vol. 36, p. 7305-7308; and others. For example, exemplary compound (B-1) is prepared in reaction of exemplary compound (M-1) with terephthaloyl chloride. Alternatively, exemplary compound (B-2) is prepared in reaction of exemplary compound (M-4) with α,α′-dibromo-m-xylene. Yet alternatively, exemplary compound (B-3) is prepared in reaction of exemplary compound (M-7) with formaldehyde. Yet alternatively, exemplary compound (B-4) is prepared in reaction of exemplary compound (M-10) with oxalyl chloride. Yet alternatively, exemplary compound (B-29) is prepared in reaction of exemplary compound (M-44) with formaldehyde. Yet alternatively, exemplary compound (B-84) is prepared in reaction of exemplary compound (M-84) with 1,4-dibromobutane.

Hereinafter, specific examples of the compounds represented by Formula (6) above will be described below, but the present invention is not restricted thereby.

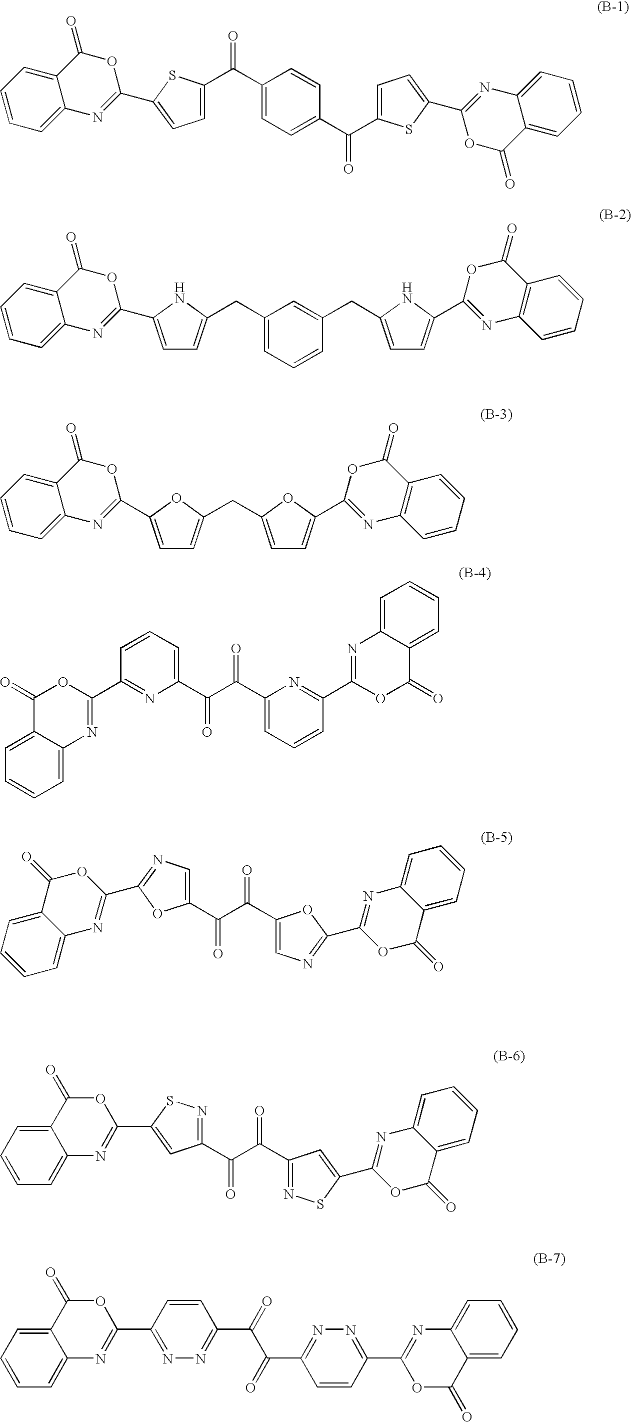

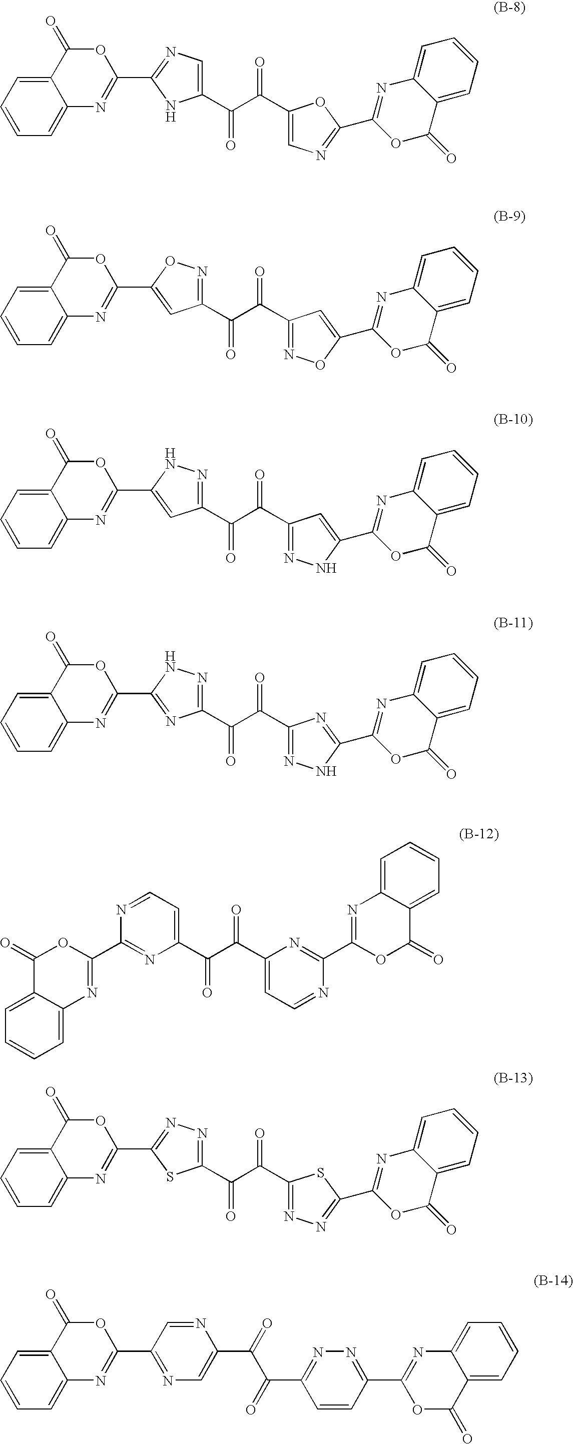

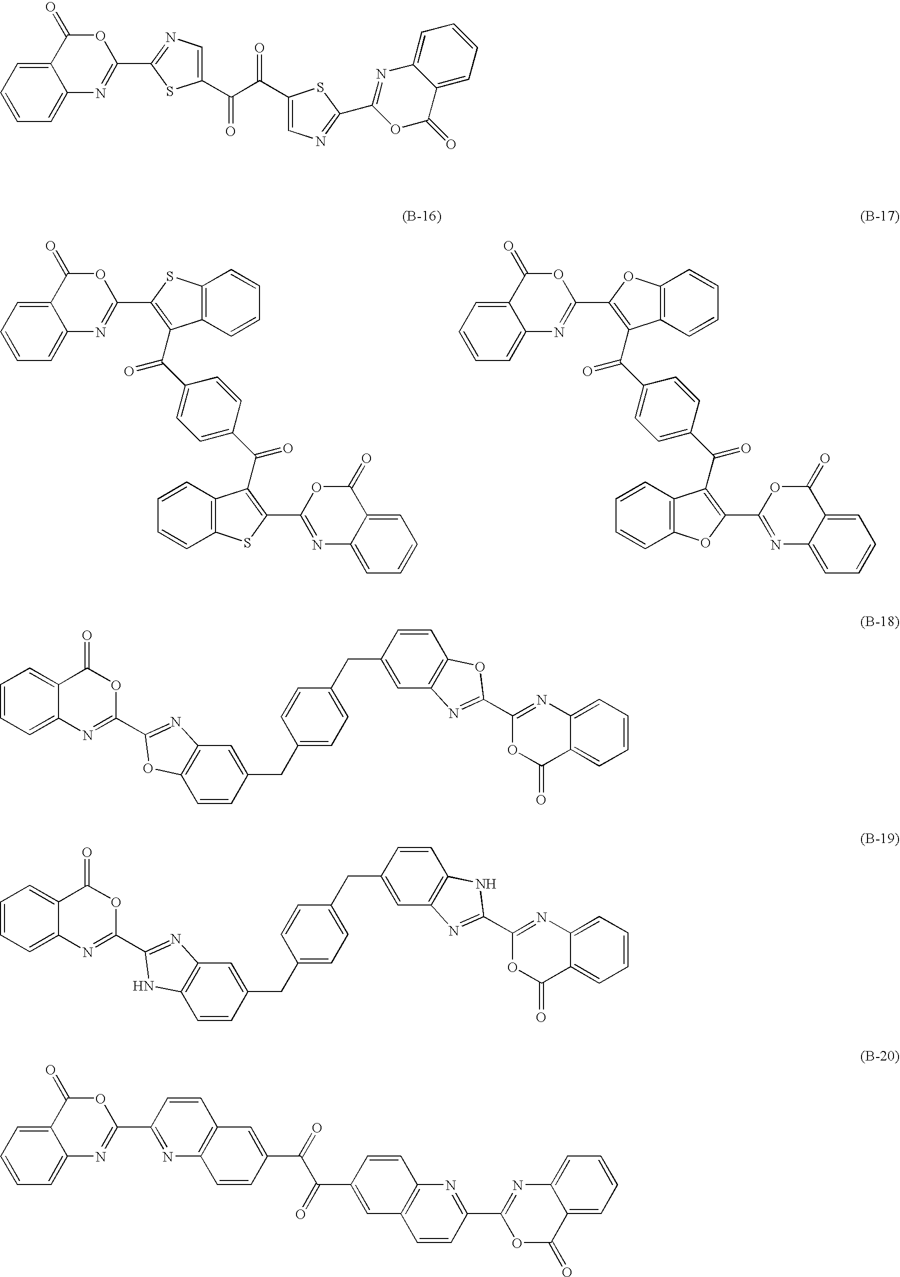

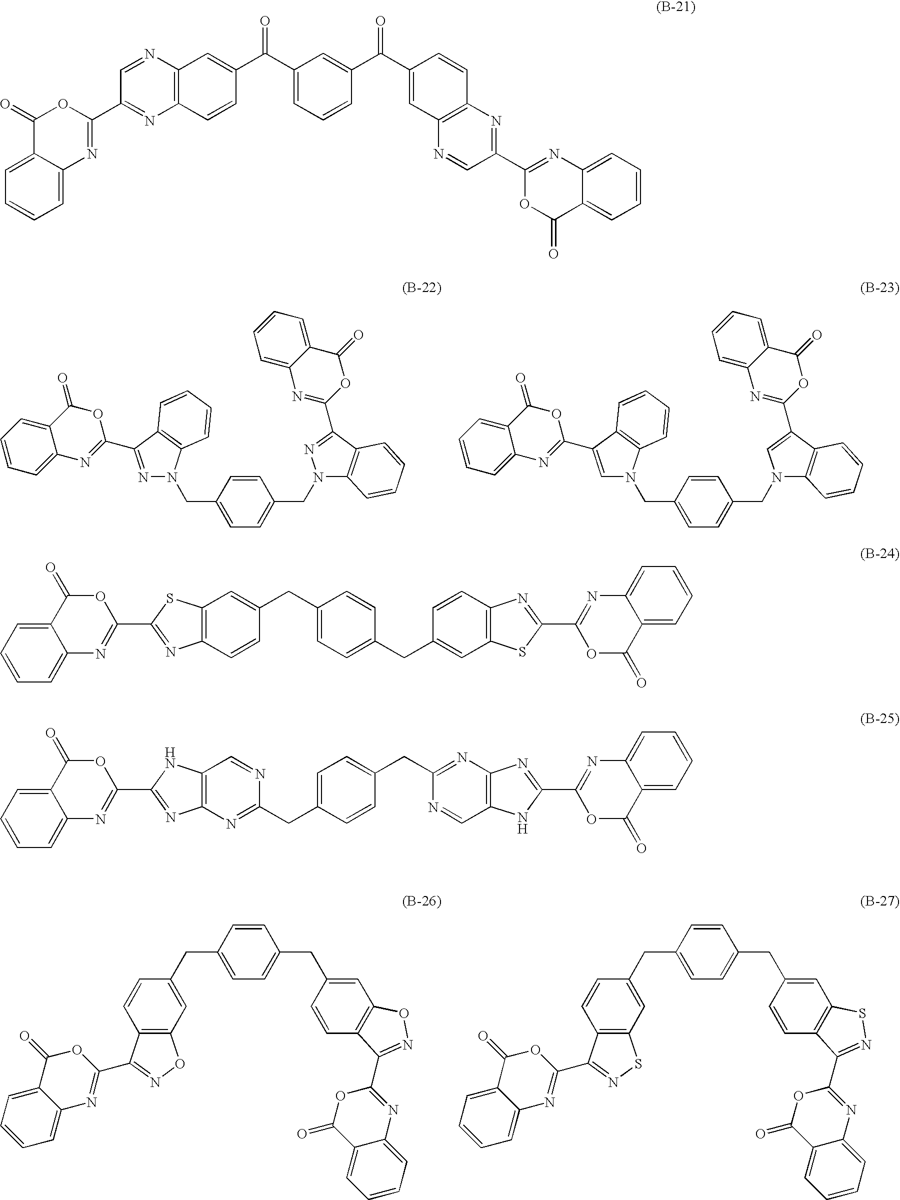

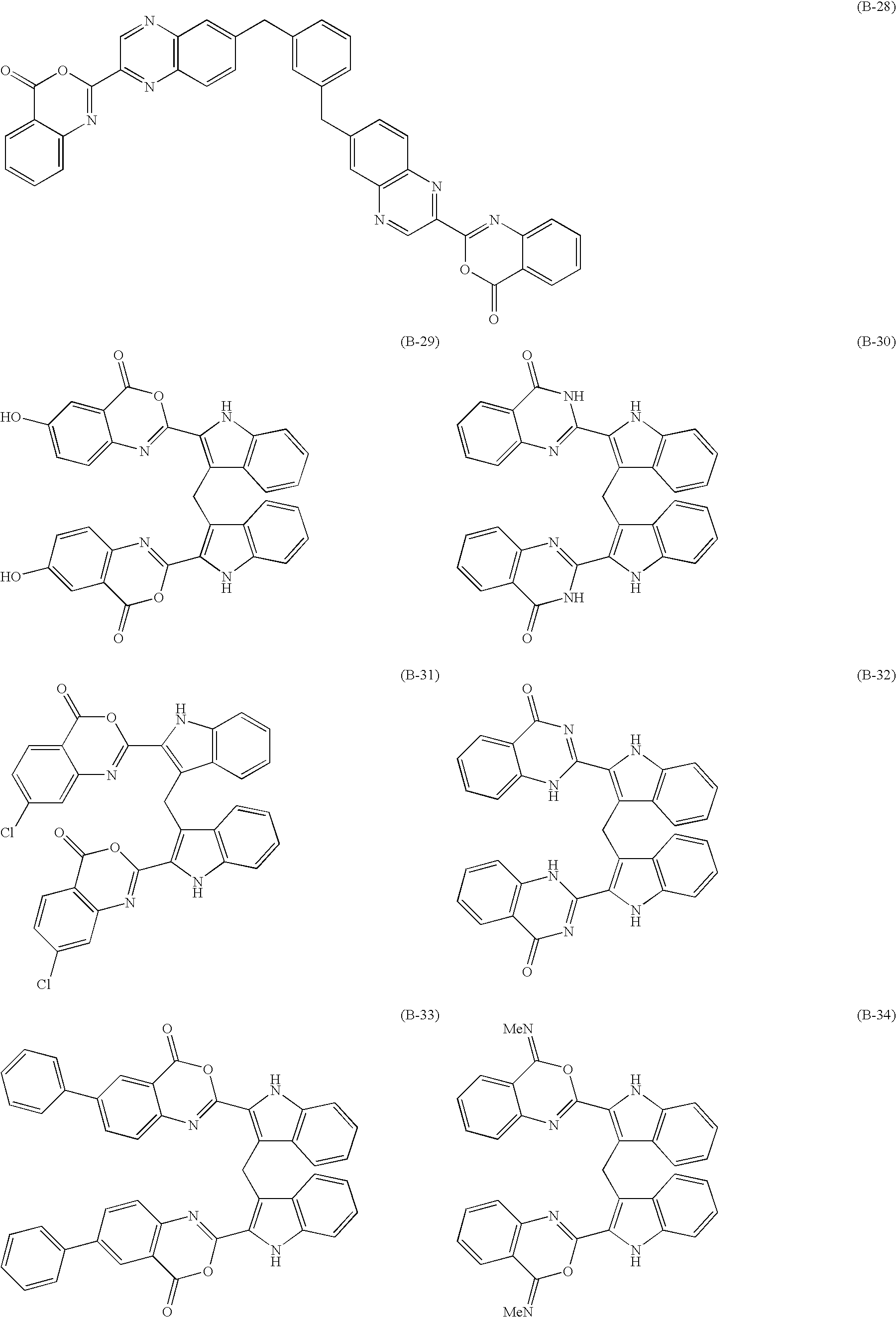

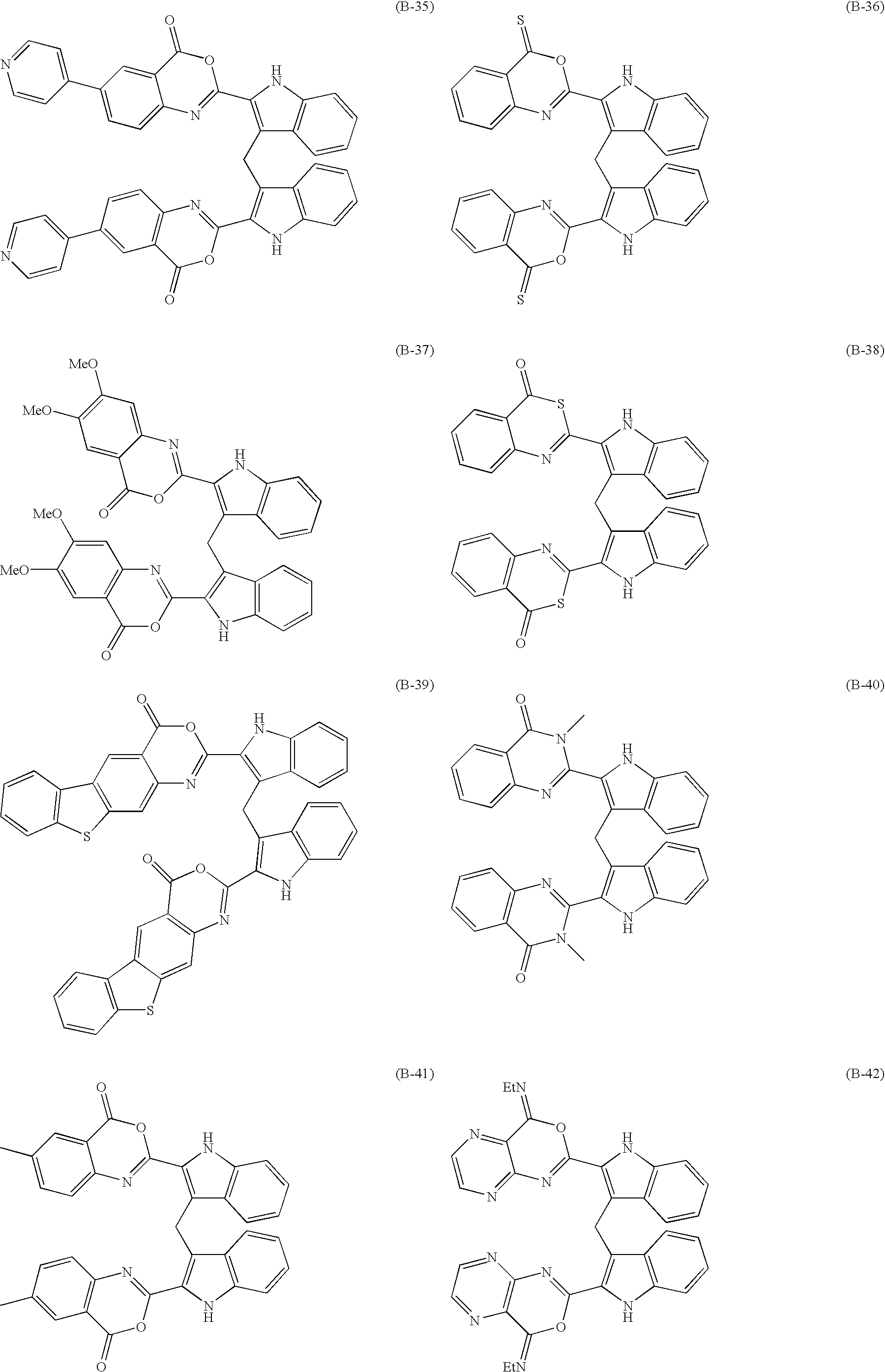

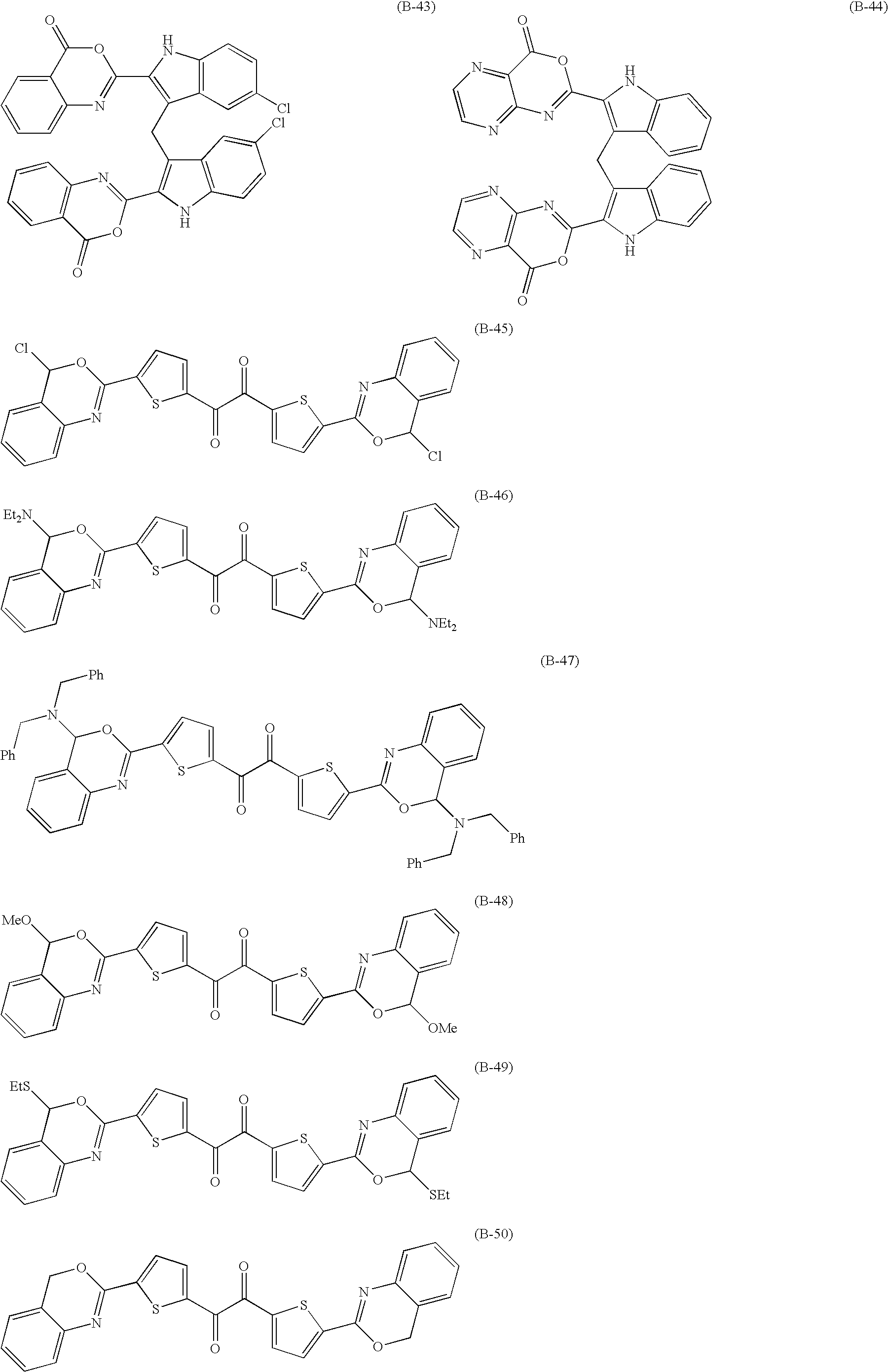

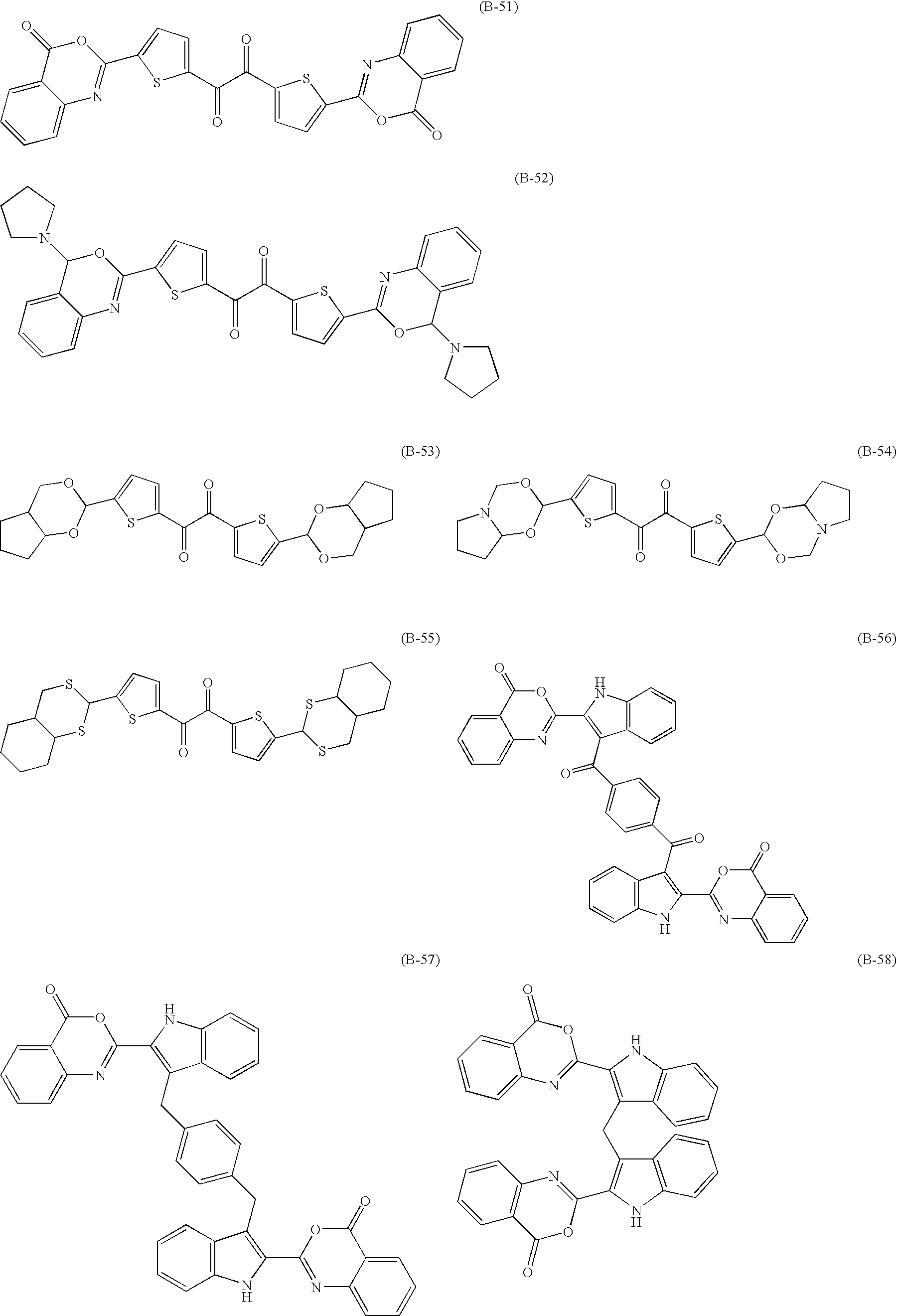

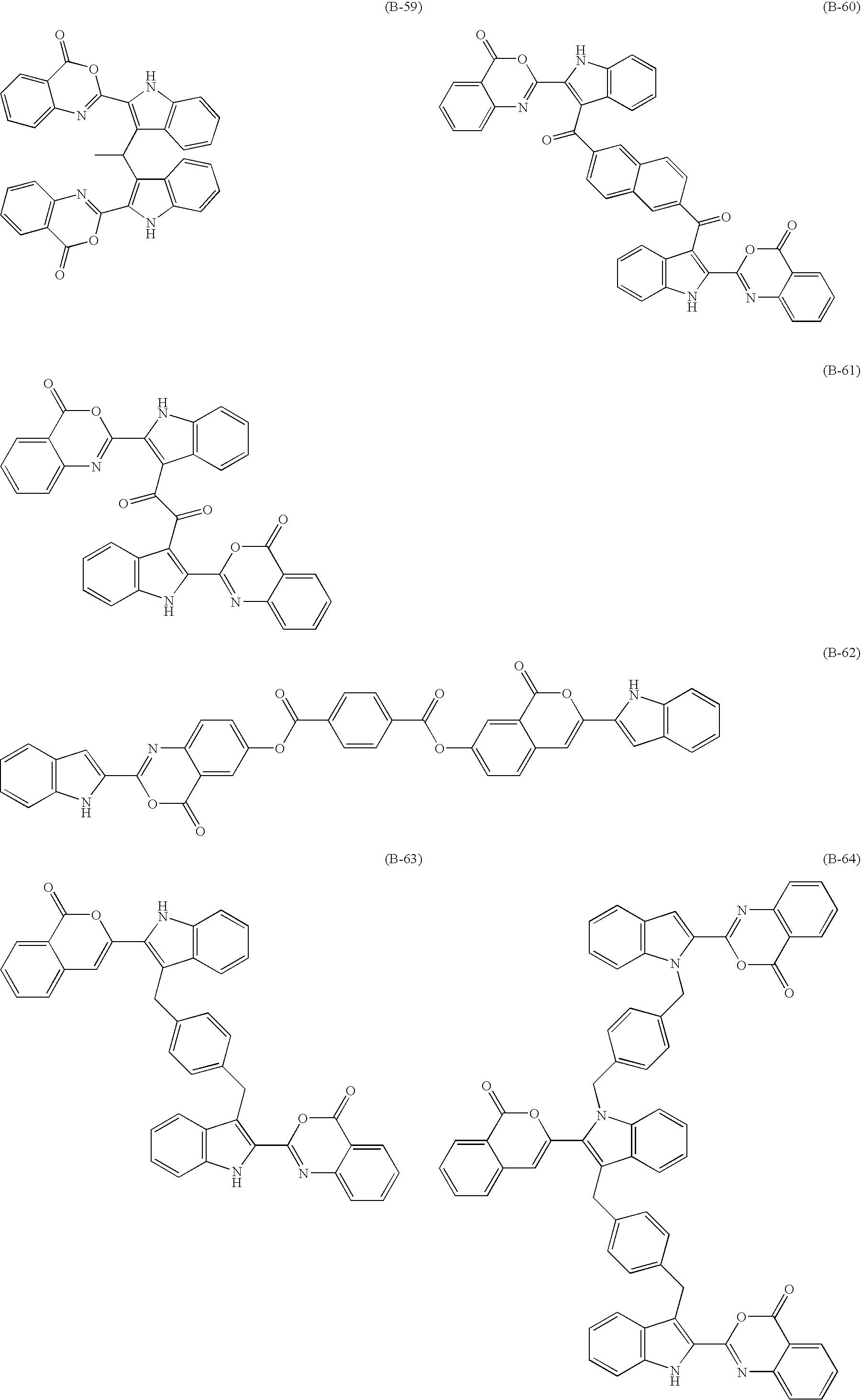

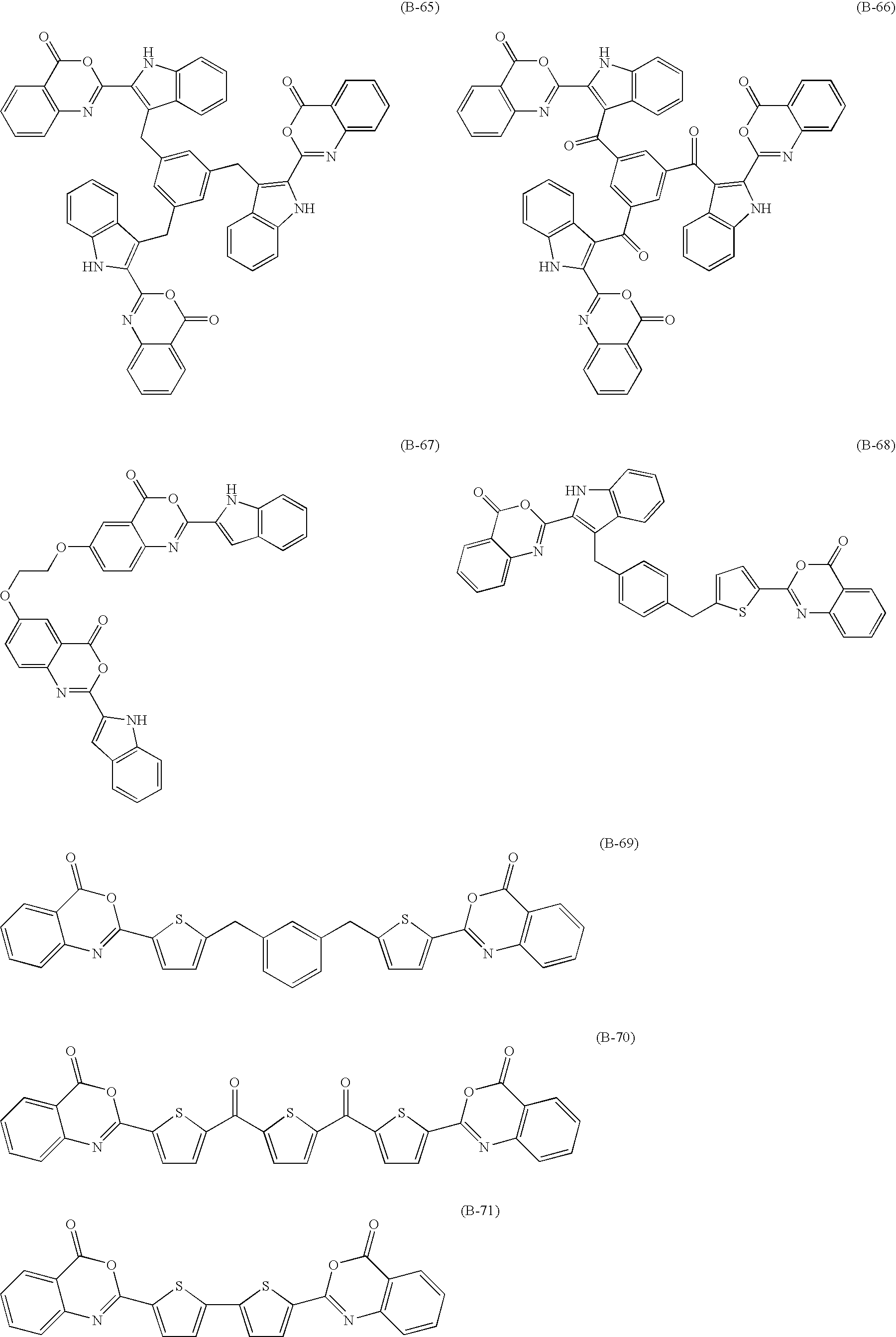

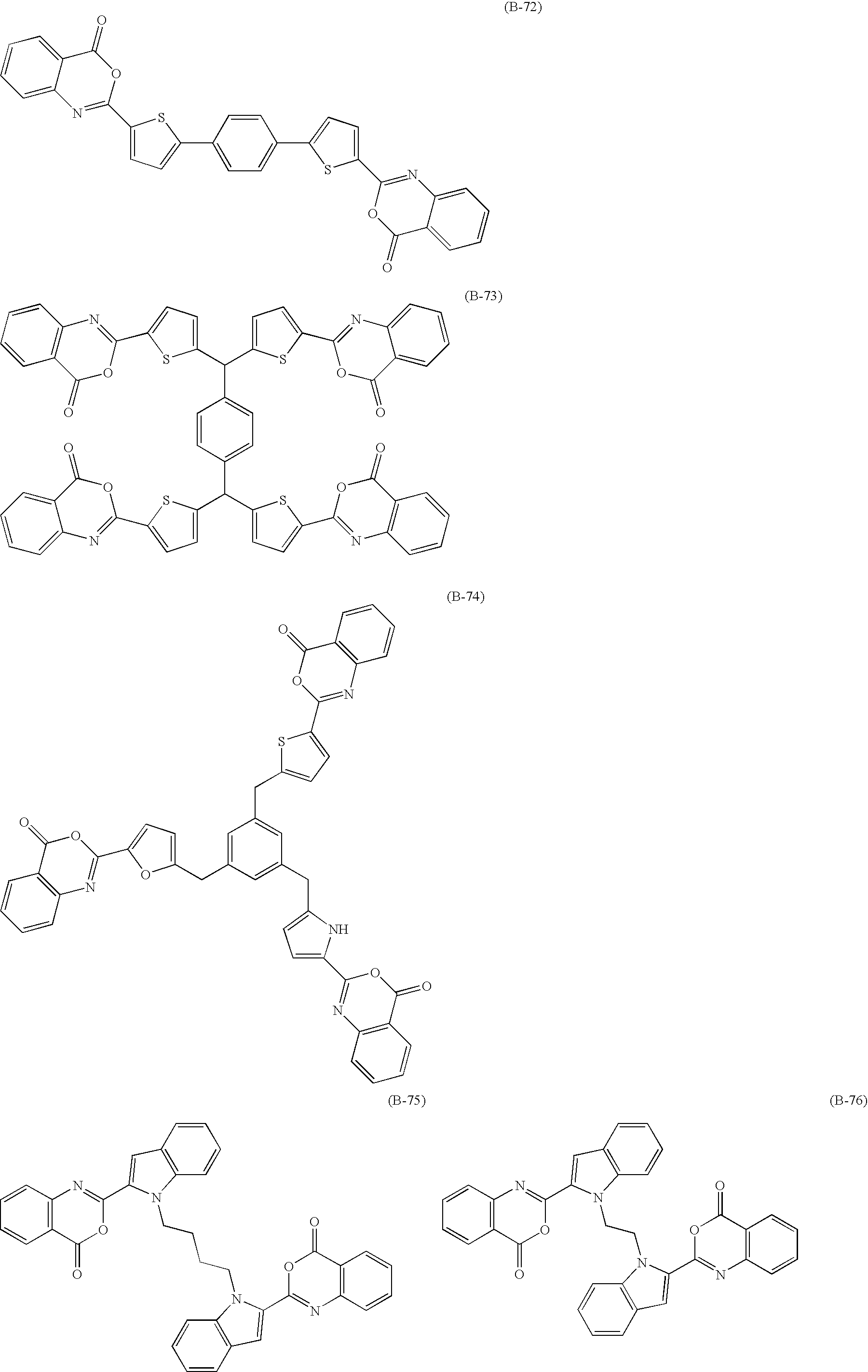

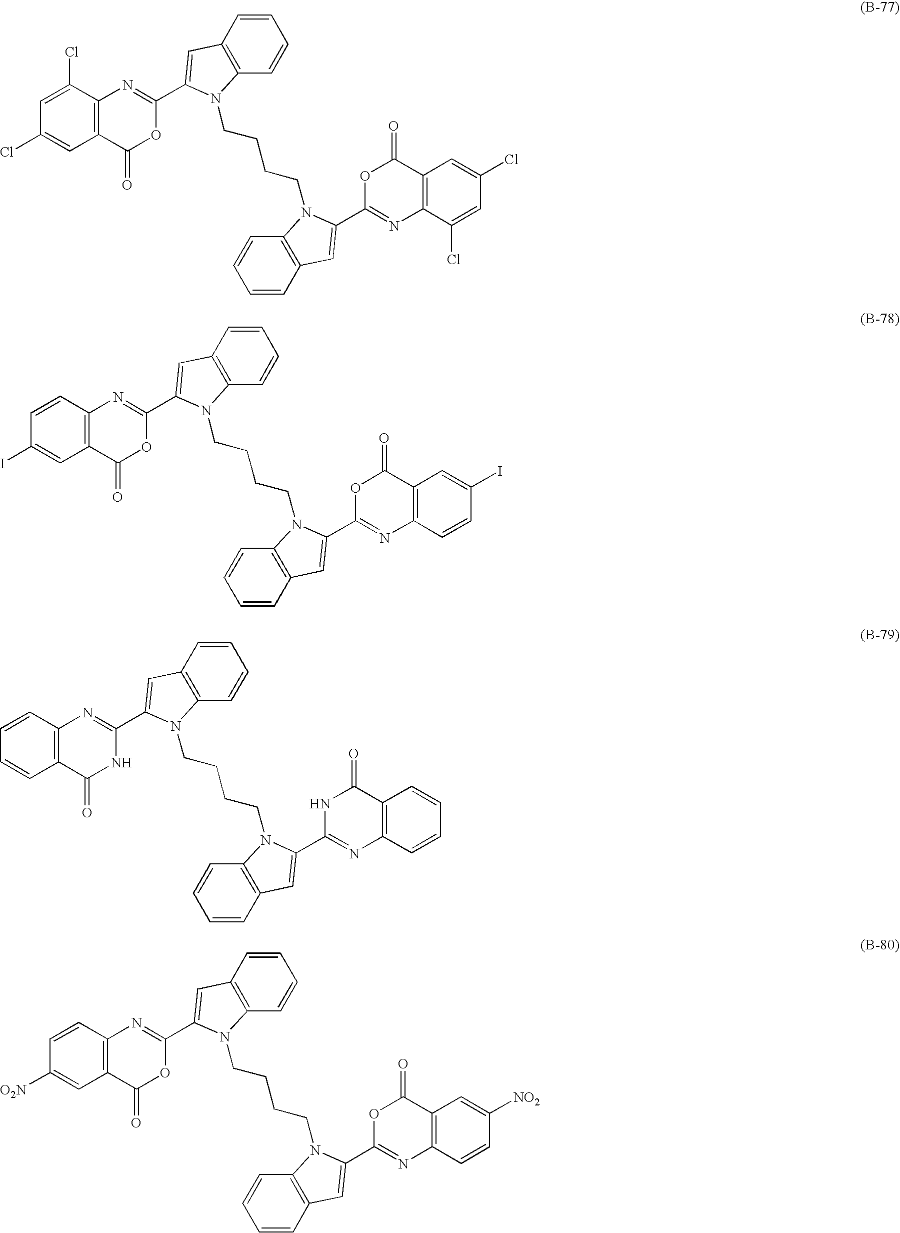

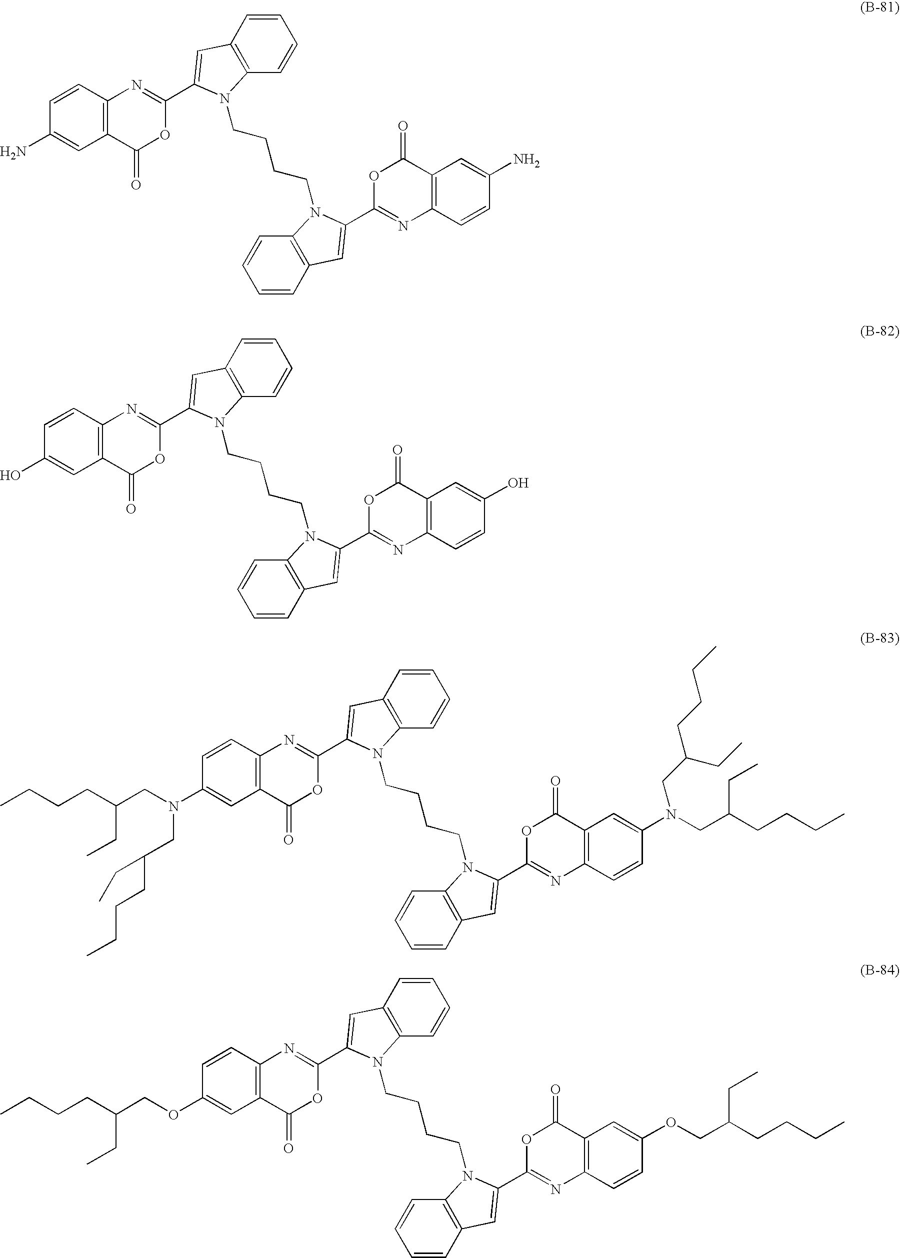

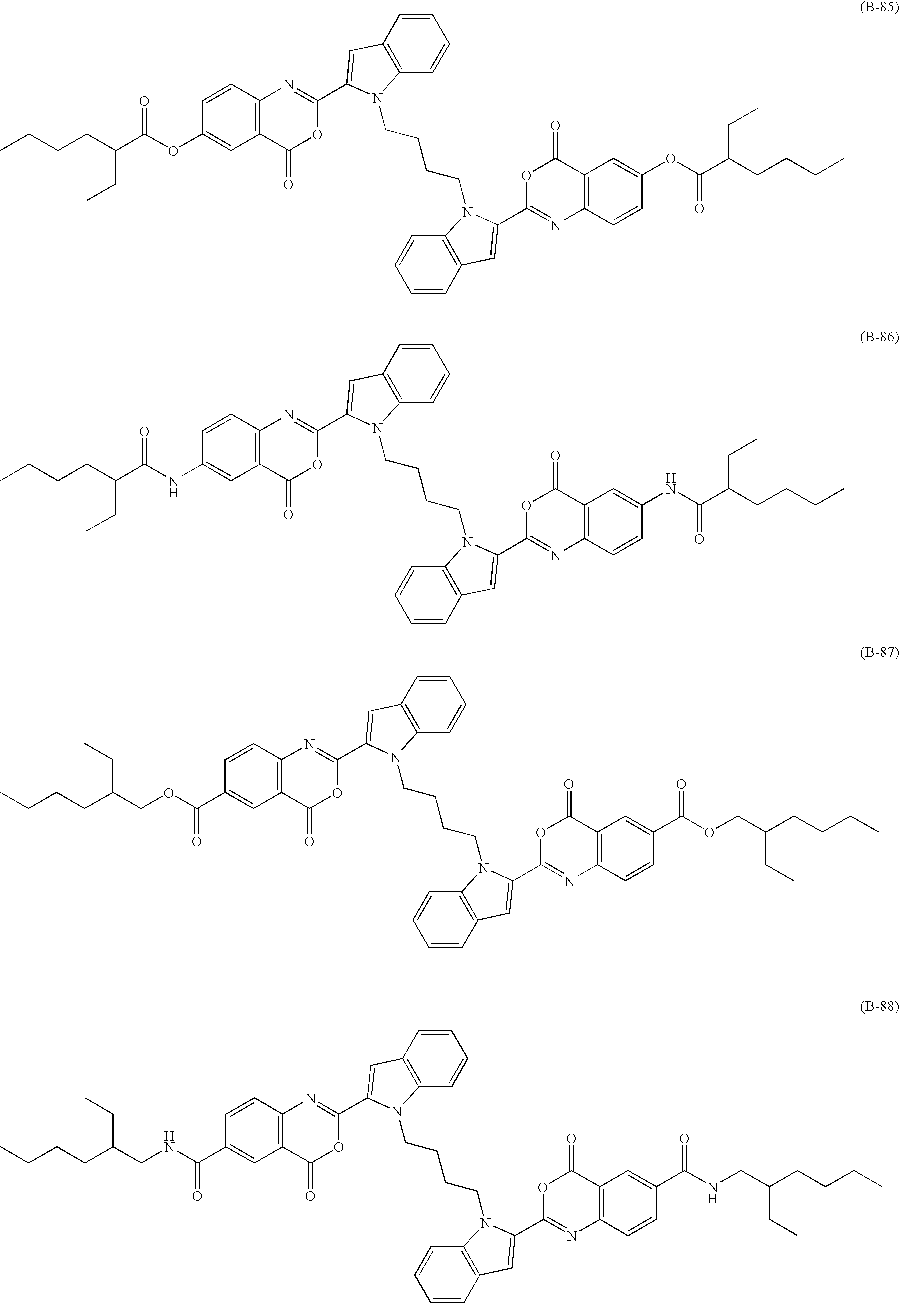

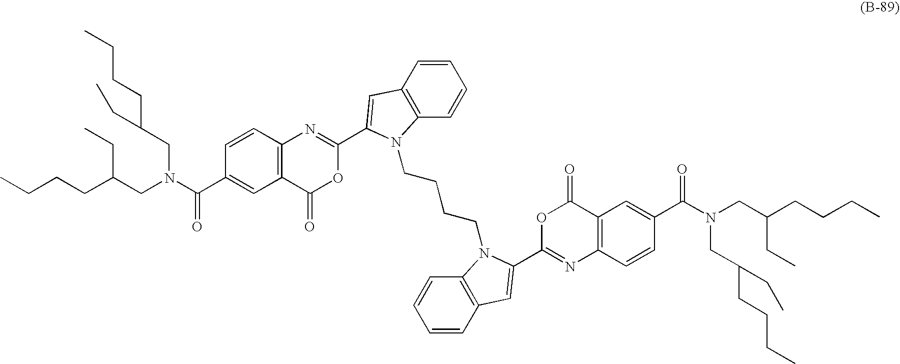

The compound represented by any one of Formulae (1) to (10) above may have tautomers depending on the structure and the environment where the compound is located. A typical form thereof is described here in the present invention, but the tautomers different from that described in the present invention are also included in the compound according to the present invention.

The compound represented by any one of Formulae (1) to (10) above may have an isotopic element (such as ²H, ³H, ¹³C, ¹⁵N, ¹⁷O, or ¹⁸O).

A polymer having the structure of the compound represented by Formulae (1) to (10) above in its recurring unit can also be used favorably in the present invention. The polymer may be a homopolymer having a single recurring unit or a copolymer having two or more kinds of recurring units. It may be a copolymer having another recurring unit additionally. Examples of the polymers having an ultraviolet absorbent structure in the recurring unit are described, for example, in JP-B-1-53455 (“JP-B” means examined Japanese patent publication), JP-A-61-189530 and EP Patent No. 27242. The polymer can be prepared with reference to the methods described in these patent documents.

The compound represented by any one of Formulae (6) to (10) is particularly suitable for use in stabilizing an organic material against damage by light, oxygen or heat. In particular, it is favorably used as a photostabilizer, particularly favorably as an ultraviolet absorbent.

Hereinafter, the ultraviolet absorbent represented by any one of Formulae (1) to (10) above will be described.

The ultraviolet absorbent according to the present invention may be in any type of usage, for example, liquid dispersion, solution, polymer material, or the like.

The maximum absorption wavelength of the ultraviolet absorbent according to the present invention is not particularly limited, but preferably 250 to 400 mm, more preferably 280 to 380 nm.

The ultraviolet absorbent according to the present invention represented by any one of Formulae (1) to (10) above can be used in the dispersed state as dispersed in a dispersed medium. Hereinafter, the ultraviolet absorbent dispersion including the ultraviolet absorbent according to the present invention will be described.

The medium for dispersing the ultraviolet absorbent according to the present invention is arbitrary. Examples thereof include water, organic solvents, resins, resin solutions, and the like. These media may be used alone or in combination of two or more.

Examples of the organic solvents as the dispersed medium for use in the present invention include hydrocarbon-based solvents such as pentane, hexane, and octane; aromatic solvents such as benzene, toluene, and xylene; ether-based solvents such as diethylether and methyl-t-butylether; alcoholic solvents such as methanol, ethanol, and isopropanol; ester-based solvents such as acetone, ethyl acetate and butyl acetate; ketone-based solvents such as methyl ethyl ketone; nitrile-based solvents such as acetonitrile and propionitrile; amide-based solvents such as N,N-dimethylformamide, N,N-dimethylacetamide, and N-methylpyrrolidone; sulfoxide-based solvents such as dimethylsulfoxide; amine-based solvents such as triethylamine and tributylamine; carboxylic acid-based solvents such as acetic acid and propionic acid; halogen-based solvents such as methylene chloride and chloroform; heteroring-based solvents such as tetrahydrofuran and pyridine; and the like. These solvents may be used as a mixture at any rate.

Examples of the resins as the dispersed medium for use in the present invention include various known thermoplastic and thermosetting resins commonly used for production of molded article, sheet, film and others. Examples of the thermoplastic resins include polyethylene series resins, polypropylene series resins, poly(meth)acrylic ester series resins, polystyrene series resins, styrene-acrylonitrile series resins, acrylonitrile-butadiene-styrene series resins, polyvinyl chloride series resins, polyvinylidene chloride series resins, polyvinyl acetate series resins, polyvinylbutyral series resins, ethylene-vinyl acetate series copolymers, ethylene-vinylalcohol series resins, polyethylene terephthalate resins (PET), polybutylene terephthalate resins (PBT), liquid crystal polyester resins (LCP), polyacetal resins (POM), polyamide resins (PA), polycarbonate resins, polyurethane resins, polyphenylene sulfide resins (PPS) and the like, and these resins may be used alone or as polymer blend or alloy of two or more. The resin may be used as a thermoplastic molding material containing a natural resin and additionally a filler such as glass fiber, carbon fiber, semi-carbonized fiber, cellulosic fiber or glass bead, a flame retardant, and the like. As needed, resin additives traditionally used, such as polyolefin series resin fine powder, polyolefin series wax, ethylene bisamide wax, and metal soap, may be used alone or in combination.

Examples of the thermosetting resins include epoxy resins, melamine resins, unsaturated polyester resins, and the like, and the resin may be used as a thermosetting molding material containing a natural resin and additionally a filler, such as glass fiber, carbon fiber, semi-carbonized fiber or cellulosic fiber or glass bead, and a flame retardant.

The ultraviolet absorbent dispersion according to the present invention may contain other additives such as dispersant, antifoam, preservative, antifreezing agent, surfactant, and others. The dispersion may contain any other compounds additionally. Examples of the other additives include dye, pigment, infrared absorbent, flavoring agent, polymerizable compound, polymer, inorganic material, metal and the like.

For example, a high-shearing force high-speed-agitation dispersing machine or a high-strength ultrasonic dispersing machine may be used as the apparatus for preparation of the ultraviolet absorbent dispersion according to the present invention. Specific examples thereof include colloid mill, homogenizer, capillary emulsifier, liquid siren, electromagnetic-distortion ultrasonic wave generator, emulsifier having a Pallmann whistle, and the like. The high-speed-agitation dispersing machine favorably used in the present invention is a dispersing machine in which a dispersing part is revolving in solution at high speed (500 to 15,000 rpm, preferably 2,000 to 4,000 rpm) such as dissolver, polytron, homomixer, homoblender, keddy mill, or jet agitator. The high-speed-agitation dispersing machine for use in the present invention is also called a dissolver or a high-speed impeller dispersing machine, and, as described in JP-A-55-129136, a dispersing machine having impellers of saw-teeth shaped plate alternately bent in the vertical direction that are connected to the shaft revolving at high speed is also a favorable example.

Various methods may be used in preparation of an emulsified dispersion containing a hydrophobic compound. For example, in dissolving a hydrophobic compound in an organic solvent, the hydrophobic compound is dissolved in a solvent or a mixture of two or more selected from high-boiling point organic materials, water-immiscible low boiling point organic solvents and water-miscible organic solvents, and the solution is then dispersed in water or an aqueous hydrophilic colloid solution in the presence of a surfactant compound. The water-insoluble phase containing the hydrophobic compound and the aqueous phase may be mixed by the so-called normal mixing method of adding the water-insoluble phase into the agitated aqueous phase or by the reverse mixing method of adding the phases reversely.

The ultraviolet absorbent according to the present invention is favorably used in the state of a solution dissolved in a liquid medium. Hereinafter, the ultraviolet absorbent solution according to the present invention will be described.

The liquid dissolving the ultraviolet absorbent according to the present invention is arbitrary. It is, for example, water, an organic solvent, a resin, a resin solution, or the like. Examples of the organic solvent, the resin, and the resin solution include those described above as the dispersed medium. These may be used alone or in combination.

The solution of the ultraviolet absorbent according to the present invention may contain any other compounds additionally. Examples of the other additives include dye, pigment, infrared absorbent, flavoring agent, polymerizable compound, polymer, inorganic material, metal and the like. Components other than the ultraviolet absorbent according to the present invention may not necessarily be dissolved.

The content of the ultraviolet absorbent in the ultraviolet absorbent solution according to the present invention may not be determined specifically, because it varies according to application and type of usage, and thus the concentration is arbitrary according to application. The concentration in the entire solution is preferably 0.001 to 30 mass %, more preferably 0.01 to 10 mass %. A solution at higher concentration may be prepared and diluted at a desired time before use. The dilution solvent is selected arbitrarily from the solvents described above.

Examples of the materials stabilized by the ultraviolet absorbent according to the present invention include dyes, pigments, foods, beverages, body-care products, vitamins, pharmaceuticals, inks, oils, fats, waxes, surface coating agents, cosmetics, photographic materials, fabrics and the dyes thereof, plastic materials, rubbers, paints, polymer materials, polymer additives and the like.

The ultraviolet absorbent according to the present invention may be used by any method when used. The ultraviolet absorbents according to the present invention may be used alone, or used as a composition, but are preferably used as a composition. In particular, polymer materials containing the ultraviolet absorbent according to the present invention are favorable. Hereinafter, the polymer materials containing the ultraviolet absorbent according to the present invention will be described.

The polymer material containing the ultraviolet absorbent according to the present invention contains a polymer substance. The polymer material containing the ultraviolet absorbent according to the present invention may be a material only of a polymeric substance or a solution of a polymer substance in any solvent.

The ultraviolet absorbent according to the present invention is added to the polymer substance in various methods. If the ultraviolet absorbent according to the present invention is compatible with the polymer substance, the ultraviolet absorbent according to the present invention may be added to the polymer substance directly. The ultraviolet absorbent according to the present invention may be dissolved in a cosolvent compatible with the polymer substance, and then the solution be added to the polymer substance. The ultraviolet absorbent according to the present invention may be dispersed in a high-boiling point organic solvent or a polymer, and the dispersion be added to the polymer substance.

The boiling point of high-boiling point organic solvent is preferably 180° C. or higher, more preferably 200° C. or higher. The melting point of the high-boiling point organic solvent is preferably 150° C. or lower, more preferably 100° C. or lower. Examples of the high-boiling point organic solvents include phosphoric esters, phosphonic esters, benzoic esters, phthalic esters, fatty acid esters, carbonate esters, amides, ethers, halogenated hydrocarbons, alcohols and paraffins. Phosphoric esters, phosphonic esters, phthalic ester, benzoic esters and fatty acid esters are preferable.

The method of adding the ultraviolet absorbent according to the present invention is determined, by reference to the description in JP-A-58-209735, JP-A-63-264748, JP-A-4-191851, and JP-A-8-272058, and British Patent No. 2016017A.

The content of the ultraviolet absorbent according to the present invention in the ultraviolet absorbent solution is not determined specifically, because it varies according to application and type of usage, and the concentration is arbitrary according to desirable application. It is preferably 0.001 to 10 mass %, more preferably 0.01 to 5 mass %, in the polymer material.

Although practically sufficient ultraviolet-shielding effect is obtained only with the ultraviolet absorbent according to the present invention in the present invention, a white pigment which has higher hiding power such as titanium oxide may be used for assurance. In addition, a trace (0.05 mass % or less) amount of colorant may be used additionally, if the appearance or the color tone is of a problem or as needed. Alternatively, a fluorescent brightener may be used additionally for applications demanding transparency or whiteness. Examples of the fluorescent brighteners include commercialized products, the compounds represented by Formula [1] and typical exemplary compounds 1 to 35 described in JP-A-2002-53824, and the like.

Hereinafter, the polymer substance for use in the present invention will be described. The polymer substance may be a natural or synthetic polymer. Examples thereof include polyolefins (such as polyethylene, polypropylene, polyisobutylene, poly(1-butene), poly-4-methylpentene, polyvinylcyclohexane, polystyrene, poly(p-methylstyrene), poly(α-methylstyrene), polyisoprene, polybutadiene, polycyclopentene, and polynorbornene); copolymers of a vinyl monomer (such as ethylene/propylene copolymer, ethylene/methylpentene copolymer, ethylene/heptene copolymer, ethylene/vinylcyclohexane copolymer, ethylene/cycloolefin copolymer (e.g., cycloolefin copolymer such as ethylene/norbornene), propylene/butadiene copolymer, isobutylene/isoprene copolymer, ethylene/vinylcylcohexene copolymer, ethylene/alkyl acrylate copolymer, and ethylene/alkyl methacrylate copolymer); acrylic polymers (such as polymethacrylate, polyacrylate, polyacrylamide, and polyacrylonitrile); polyvinyl chloride, polyvinylidene chloride, polyvinyl fluoride, polyvinylidene fluoride, vinyl chloride/vinyl acetate copolymer, polyethers (such as polyalkylene glycol, polyethyleneoxide, and polypropyleneoxide); polyacetals (such as polyoxymethylene); polyamide, polyimide, polyurethane, polyurea, polyesters (such as polyethylene terephthalate and polyethylene naphthalate); polycarbonate, polyketone, polysulfone polyether ketone, phenol resins, melamine resins, cellulose esters (such as diacetylcellulose, triacetylcellulose (TAC), propionylcellulose, butyrylcellulose, acetyl propionylcellulose, and nitrocellulose); polysiloxane, natural polymers (such as cellulose, rubber, and gelatin), and the like.

The polymer substance for use in the present invention is preferably a synthetic polymer, more preferably a polyolefin, an acrylic polymer, polyester, polycarbonate, or a cellulose ester. Among them, polyethylene, polypropylene, poly(4-methylpentene), polymethyl methacrylate, polycarbonate, polyethylene terephthalate, polyethylene naphthalate, polybutylene terephthalate, and triacetylcellulose are particularly preferable.

The polymer substance for use in the present invention is preferably a thermoplastic resin.

The ultraviolet absorbent according to the present invention may be added in any desirable amount for providing desired properties. A smaller content leads to insufficient ultraviolet-shielding effect, while an excessive content to generation of the problem of bleeding out; the favorable content varies according to the compound and the polymer substance used, but is determined properly in experiment by those who are skilled in the art. The content thereof in the polymer material is preferably more than 0 mass % and 20 mass % or less, more preferably more than 0 mass % and 10 mass % or less, and still more preferably 0.05 mass % or more and 5 mass % or less.

The polymer material according to the present invention may contain any additives such as antioxidant, photostabilizer, processing stabilizer, antioxidant, and compatibilizer, as needed in addition to the polymer substance above and the ultraviolet light inhibitor.

The polymer material including the ultraviolet absorbent according to the present invention is applicable to any application where synthetic resin is used, and particularly favorably to applications where there is possibility of exposure to light such as sunlight or ultraviolet light. Specific examples thereof include glass alternatives and their surface-coating agent; coating agents for the window glass, lighting glass and light-protecting glass such as of house, facility, and vehicle; window films such as of house, facility and vehicle; interior and exterior materials such as of house, facility and vehicle, paints for the interior and exterior materials, and the paint films formed by the paints; alkyd resin lacquer paints and the paint films formed by the paints; acryl lacquer paints and the paint films formed by the paints; materials for ultraviolet-emission sources such as fluorescent lamp and mercury lamp; materials for precision machines and electric and electronic devices; materials for shielding electromagnetic and other waves emitted from various displays; containers or packaging materials for foods, chemicals and drugs; special packages such as bottle, box, blister, and cup; discoloration inhibitors for compact disk coating, agricultural and industrial sheet or film, print, colored products, dyes and pigments; protective film for polymer supports (e.g., plastic parts such as mechanical and automotive parts); print over-coating, inkjet medium film, delustered laminate film, optical light film, safety glass/front glass intermediate layer, electrochromic/photochromic film, over-lamination film, solar-heat-controlling film, cosmetics such as anti-sunburn cream, shampoo, rinse, and hair dressing; apparel fiber products such as sport wear, stockings and cap and the fibers; home interior products such as curtain, carpet and wall paper; medical devices such as plastic lens, contact lens and artificial eye; optical materials such as optical filter, backlight display film, prism, mirror, and photographic material; mold film, transfer-type sticker, anti-graffiti film, stationery products such as tape and ink; display plates and devices and the surface-coating agents thereof, and the like.

The shape of the polymer material according to the present invention may be flat film, powder, spherical particle, crushed particle, bulky continuous particle, fiber, solenoid, hollow fiber, granule, plate, porous particle, or the other.

The polymer material according to the present invention, which contains the ultraviolet absorbent according to the present invention, is superior in light stability (ultraviolet fastness), causing no precipitation or bleed out of the ultraviolet absorbent during long-term use. In addition, the polymer material according to the present invention, which has superior long-wavelength ultraviolet absorption capacity, can be used as an ultraviolet-absorbing filter or container, for protection, for example, of an ultraviolet-sensitive compound therein. It is possible to obtain a molded article (such as container) of the polymer material according to the present invention, for example, by molding the polymer substance by any molding method such as extrusion molding or injection molding. It is also possible to prepare a molded article having an ultraviolet-absorbing film coated on the polymer material according to the present invention, by coating and drying a solution of the polymer substance on a separately prepared molded article.

When the polymer material according to the present invention is used as an ultraviolet-absorbing filter or film, the polymer substance is preferably transparent. Examples of the transparent polymer materials include cellulose esters (such as diacetylcellulose, triacetylcellulose (TAC), propionylcellulose, butyrylcellulose, acetyl propionyl cellulose, and nitrocellulose), polyamides, polycarbonates, polyesters (such as polyethylene terephthalate, polyethylene naphthalate, polybutylene terephthalate, poly-1,4-cyclohexane dimethylene terephthalate, polyethylene-1,2-diphenoxyethane-4,4′-dicarboxylate, and polybutylene terephthalate), polystyrenes (such as syndiotactic polystyrene), polyolefins (such as polyethylene, polypropylene, and polymethylpentene), polymethyl methacrylate, syndiotactic polystyrene, polysulfones, polyether sulfones, polyether ketones, polyether imides, polyoxyethylene, and the like. Preferable are cellulose esters, polycarbonates, polyesters, polyolefins, and acrylic resins, more preferable are polycarbonates and polyesters, specifically preferable is polyester, and most preferable is polyethylene terephthalate. The polymer material according to the present invention may be used as a transparent support, and the transmittance of the transparent support in such a case is preferably 80% or more, more preferably 86% or more.

In the present invention, two or more kinds of compounds represented by any one of Formulae (1) to (10) different in structure may be used in combination. Alternatively, the compound represented by any one of Formulae (1) to (10) and one or more kinds of ultraviolet absorbents different in structure may be used in combination. Two kinds (preferably three kinds) of ultraviolet absorbents when used in combination absorb ultraviolet ray in a wider wavelength range. In addition, the use of two or more kinds of ultraviolet absorbents in combination has a function to stabilize the dispersion state. The ultraviolet absorbent having a structure other than that represented by Formula (1) or (6) is not particularly limited. Examples thereof include ultraviolet absorbing structures such as triazine-based, benzotriazole-based, benzophenone-based, merocyanine-based, cyanine-based, dibenzoylmethane-based, cinnamic acid-based, cyanoacrylate-based, and benzoic ester-based compounds. Examples thereof include the ultraviolet absorbents described, for example, in Fine Chemical, May 2004, p. 28 to 38; Survey and Research Dept., Toray Research Center Inc. Ed., “Trend of Functional Additives for Polymers” (Toray Research Center Inc., 1999) p. 96 to 140; and Yasuichi Okatsu Ed., “Development of polymer additives and Environmental Measures” (CMC Publishing, 2003) p. 54 to 64.

Examples of the ultraviolet absorbent having a structure other than that represented by Formula (1) or (6) include compounds such as benzotriazole-based, benzophenone-based, salicylic-acid-based, cyanoacrylate-based, and triazine-based compounds. Particularly preferable are benzotriazole-based compounds.

The effective absorption wavelength of benzotriazole-based compounds is approximately 270 to 380 nm, and specific examples thereof include 2-(2′-hydroxy-5′-methylphenyl)benzotriazole, 2-(2′-hydroxy-5′-t-butylphenyl)benzotriazole, 2-(2′-hydroxy-3′-t-butyl-5′-methylphenyl)-5-chlorobenzotriazole, 2-(2′-hydroxy-3′,5′-di-t-butylphenyl)-5-chlorobenzotriazole, 2-(2′-hydroxy-3′-t-butyl-5′-(2-(octyloxycarbonyl)ethyl)phenyl)-5-chlorobenzotriazole, 2-(2′-hydroxy-3′-dodecyl-5′-methylphenyl)-5-chlorobenzotriazole, 2-(2′-hydroxy-3′,5′-di-t-amylphenyl)benzotriazole, 2-(2′-hydroxy-5′-t-octylphenyl)benzotriazole, 2-(2′-hydroxy-3′,5′-di-(dimethylbenzyl)phenyl)benzotriazole, 2-(2′-hydroxy-4′-octyloxyphenyl)benzotriazole, 2,2′-methylene-bis(2-(2′-hydroxy-5′-t-octylphenyl)benzotriazole)-2-(2′-hydroxy-3′-(3,4,5,6-tetrahydrophthalimidylmethyl)-5′-methylbenzyl)phenyl)benzotriazole, and the like.

The effective absorption wavelength of benzophenone-based compounds is approximately 270 to 380 nm, and specific examples thereof include 2,4-dihydroxybenzophenone, 2-hydroxy-4-methoxybenzophenone, 2-hydroxy-4-octyloxybenzophenone, 2-hydroxy-4-decyloxybenzophenone, 2-hydroxy-4-benzyloxybenzophenone, 2-hydroxy-4-(2-hydroxy-3-methacryloxypropoxy)benzophenone, 2-hydroxy-4-methoxy-5-sulfobenzophenone, 2-hydroxy-4-methoxy-5-sulfobenzophenone trihydrate, 2-hydroxy-4-methoxy-2′-carboxybenzophenone, 2-hydroxy-4-octadecyloxybenzophenone, 2-hydroxy-4-diethylamino-2′-hexyloxycarbonylbenzophenone, 2,2′-dihydroxy-4-methoxybenzophenone, 2,2′,4,4′-tetrahydroxybenzophenone, 2,2′-dihydroxy-4,4′-dimethoxybenzophenone, 1,4-bis(4-benzyloxy-3-hydroxyphenoxy)butane, and the like.

The effective absorption wavelength of the salicylic-acid-based compounds is approximately 290 to 330 nm, and typical examples thereof include phenyl salicylate, p-t-butylphenyl salicylate, p-octylphenyl salicylate, and the like.

The effective absorption wavelength of cyanoacrylate-based compounds is approximately 270 to 350 nm, and specific examples thereof include 2-ethylhexyl 2-cyano-3,3-diphenylacrylate, ethyl 2-cyano-3,3-diphenylacrylate, hexadecyl 2-cyano-3-(4-methylphenyl)acrylate, salt of 2-cyano-3-(4-methylphenyl)acrylic acid, 1,3-bis(2′-cyano-3,3′-diphenylacryloyl)oxy)-2,2-bis(((2′-cyano-3,3′-diphenylacryloyl)oxy)methyl)propane, and the like.

The effective absorption wavelength of the triazine compounds is approximately 270 to 380 nm, and specific examples thereof include 2-(4-hexyloxy-2-hydroxyphenyl)-4,6-diphenyl-1,3,5-triazine, 2-(4-octyloxy-2-hydroxyphenyl)-4,6-di(2,5-dimethylphenyl)-1,3,5-triazine, 2-(4-butoxy-2-hydroxyphenyl)-4,6-di(4-butoxyphenyl)-1,3,5-triazine, 2-(4-butoxy-2-hydroxyphenyl)-4,6-di(2,4-dibutoxyphenyl)-1,3,5-triazine, 2-(4-(3-(2-ethylhexyloxy)-2-hydroxypropoxy)-2-hydroxyphenyl)-4,6-di(2,4-dimethylphenyl)-1,3,5-triazine, 2-(4-(3-dodecyloxy-2-hydroxypropoxy)-2-hydroxyphenyl)-4,6-di(2,4-dimethylphenyl)-1,3,5-triazine, 2,4-di(4-butoxy-2-hydroxyphenyl)-6-(4-butoxyphenyl)-1,3,5-triazine, 2,4-di(4-butoxy-2-hydroxyphenyl)-6-(2,4-dibutoxyphenyl)-1,3,5-triazine, and the like.

The compound represented by any one of Formulae (1) to (10) can be used favorably as a fluorescent brightener. Generally, fluorescent brighteners are compounds absorbing light at a wavelength of approximately 320 to approximately 410 nm and emitting light at a wavelength of approximately 410 to approximately 500 nm. Fabrics dyed with such a fluorescent brightener give blue light at a wavelength of approximately 410 to approximately 500 nm newly emitted from the fluorescent brightener in addition to its inherent yellow reflection light, giving white reflection light in combination, and increase in visible light energy by fluorescent effect results in increase in whiteness in appearance.

Hereinafter, fluorescent brighteners of the compounds represented by any one of Formulae (1) to (10) will be described. The fluorescent brightener according to the present invention may be used in any form of product. For example, it may be used as a liquid dispersion, a solution, a polymer material, or the like.

The fluorescent brightener above may be used as it is dispersed in a dispersion medium. The dispersion medium, preparative process, fluorescent brightener content and dispersion apparatus for the dispersion containing the fluorescent brightener above are the same as those for the ultraviolet absorbent described above.

Alternatively, the fluorescent brightener above may be used as it is dissolved in a liquid medium. The addition method of the fluorescent brightener above to the solution, the content thereof, and the solution are the same as those for the ultraviolet absorbent described above.

The fluorescent brightener above is used favorably in a polymer material. The polymer substance, additive, shape, application of the polymer material containing the fluorescent brightener above are the same as those described for the ultraviolet absorbent described above.

If the fluorescent brightener above by itself can not remove ultraviolet ray in the short-wavelength region sufficiently, an additional ultraviolet absorbent is preferably used.

The ultraviolet absorbent used in combination is not particularly limited. Examples thereof include ultraviolet absorbents represented by any one of Formulae (1) to (10) and the ultraviolet absorbent described above. These ultraviolet absorbents may be used alone or in combination of two or more.

The present invention provides a compound that may be used as an ultraviolet absorbent absorbing long-wavelength ultraviolet ray for an extended period of time that improves the UV light resistance of the polymer material used in combination and inhibits decomposition of the other less-stable compounds as used in the form of an ultraviolet filter.

The compound according to the present invention retains its long-wavelength ultraviolet absorption capacity over a long period of time and can be used as an ultraviolet absorbent with favorable light fastness. Addition of the compound according to the present invention to a molded polymer product such as plastic part and fiber leads to improvement of the light stability (ultraviolet durability) of the molded polymer product and also of the ultraviolet durability of the polymer material used in combination. Further, the polymer material containing the compound according to the present invention can be used as a filter or container for protecting an ultraviolet-sensitive content with its superior ultraviolet absorption capacity.

The present invention will be described in more detail based on the following examples, but the invention is not intended to be limited thereby.

### EXAMPLES

### Example 1-1

### Preparation of Exemplary Compound (M-1)

0.9 g of anthranilic acid was dissolved in 2 ml of dimethylacetamide. 1 g of 2-thiophenedicarbonyl chloride was added to the solution at room temperature. The solution was reacted at room temperature for 30 minutes, and the solid obtained was filtered and washed with water, to give 1.4 g of a synthetic intermediate A (yield: 83%).

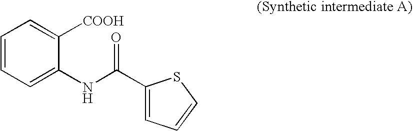

5 ml of acetic anhydride and 5 ml of toluene were added to 1.0 g of the synthetic intermediate A. The mixture was allowed to react under reflux for four hours. After cooled with iced water, the precipitate was filtered and washed with water, to give 0.7 g of an exemplary compound (M-1) (yield: 76%).

MS: m/z 230 (M+)

¹H NMR (CDCl₃): δ 7.17-7.19 (1H), δ 7.47-7.51 (1H), δ 7.61-7.65 (2H), δ 7.79-0.83 (1H), δ 7.97-7.98 (1H), δ 8.21-8.23 (1H)

λmax=309 nm (EtOAc)

### Example 1-2

### Preparation of Exemplary Compound (M-24)

2.4 g of oxalyl chloride was added dropwise to a mixture of 2.0 g of indole-2-carboxylic acid in 6 ml of toluene. After addition of a few drops of DMF, the mixture was stirred at room temperature for 30 minutes and at 80° C. for 10 minutes. The reaction solution was added to a solution of 1.7 g of anthranilic acid in 4 ml of dimethylacetamide, and the solid obtained was filtered and washed with water, to give 3.0 g of a synthetic intermediate B (yield: 86%).

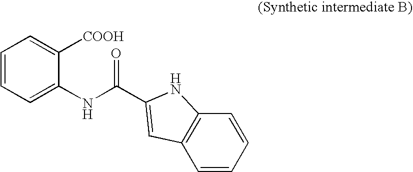

5 ml of acetic anhydride and 5 ml of toluene were added to 1.0 g of the synthetic intermediate B. The mixture was allowed to react under reflux for eight hours. After cooled with water, the precipitate was filtered and washed with water, to give 0.5 g of an exemplary compound (M-24) (yield: 53%).

MS: m/z 263 (M+)

¹H NMR (CDCl₃): δ 7.16-7.21 (1H), δ 7.33-7.38 (1H), δ 7.45-7.53 (3H), δ 7.62-7.65 (1H), δ 7.72-7.74 (1H), δ 7.80-7.85 (1H), δ 8.23-7.26 (1H)

λmax=350 nm (EtOAc)

### Example 1-3

### Preparation of Exemplary Compound (M-26)

2.4 g of oxalyl chloride was added to a mixture of 2.0 g of benzofuran-2-carboxylic acid in 6 ml of toluene. After addition of a few drops of DMF, the mixture was stirred at room temperature for 30 minutes and at 80° C. for 10 minutes. The reaction solution was added to a solution of 1.7 g of anthranilic acid in 4 ml of dimethylacetamide, and the solid obtained was filtered and washed with water, to give 3.3 g of a synthetic intermediate C (yield: 95%).

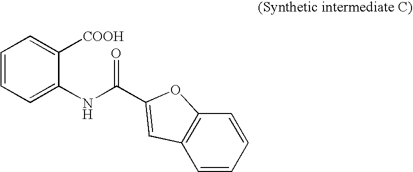

5 ml of acetic anhydride and 5 ml of toluene were added to 1.0 g of the synthetic intermediate C. The mixture was allowed to react under reflux for eight hours. After cooled with water, the precipitate was filtered and washed with water, to give 0.7 g of an exemplary compound (M-26) (yield: 75%).

MS: m/z 264 (M+)

¹H NMR (CDCl₃): δ 7.33-7.37 (1H), δ 7.46-7.50 (1H), δ 7.54-7.59 (1H), δ 7.68-7.75 (3H), δ 7.82-7.89 (2H), δ 8.26-8.28 (1H)

λmax=339 nm (EtOAc)

### <Preparation and Evaluation of Sample Solution>

One mg of exemplary compound (M-1) was dissolved in 100 ml of ethyl acetate, to give a sample solution. Similarly, sample solutions of exemplary compounds (M-24) and comparative compounds A and B respectively were prepared. The absorbance of each sample solution was determined in a 1 cm quartz cell by using Spectrophotometer UV-3600 (product name) manufactured by Shimadzu Corporation. The cell containing the sample solution was photoirradiated by a xenon lamp with its U V filter removed at an illuminance of 170,000 lux, and the amount of each compound remaining after irradiation for two days was determined. The residual amount was calculated according to the following Formula:

Residual amount (%)=100×(100−Transmittance after irradiation)/(100−Transmittance before irradiation)

The transmittance is a value determined at the maximum absorption wavelength of each compound. The result is shown in Table 16.

TABLE 16

| Sample | | Residual | |
|---|---|---|---|
| No. | Ultraviolet absorbent | amount (%) | |
| 1 | Exemplified | 96 | Present |
| | compound (M-1) | | invention |
| 2 | Exemplified | 94 | Present |
| | compound (M-24) | | invention |
| 3 | Comparative | 80 | Comparative |
| | compound A | | example |
| 4 | Comparative | 27 | Comparative |
| | compound B | | example |

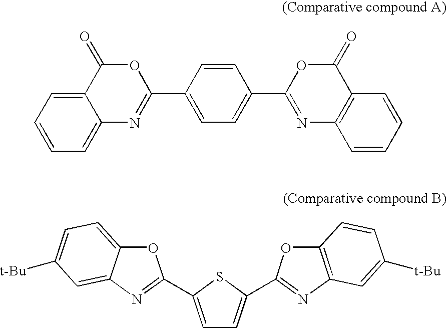

As apparent from the results in Table 16, the ultraviolet absorbents according to the present invention remained in the sample solution in an amount greater than the comparative compounds A and B (conventional ultraviolet absorbents absorbing light in the UV-A range), indicating that these compounds were more resistant to decomposition by photoirradiation.

### Example 2-1

### Preparation of Exemplary Compound (B-58)

5 ml of 37% formaldehyde was added to a mixture which was prepared by adding 300 ml of water and 3.7 ml of acetic acid to 20 g of indole-2-carboxylic acid. The mixture was stirred at 90° C. for 12 hours. The precipitate was filtered and washed with water, to give 19 g of a synthetic intermediate D (yield: 92%).

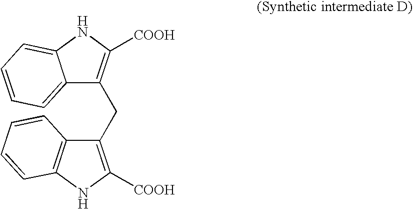

5.7 g of oxalyl chloride was added to a mixture of 5.0 g the synthetic intermediate D in 15 ml of toluene. After addition of a few drops of DMF, the mixture was stirred at room temperature for 30 minutes and at 80° C. for 10 minutes. The reaction solution was added to a solution of 4.1 g of anthranilic acid in 20 ml of dimethylacetamide, and the solid obtained was filtered and washed with water, to give 8.0 g of a synthetic intermediate E (yield: 93%).

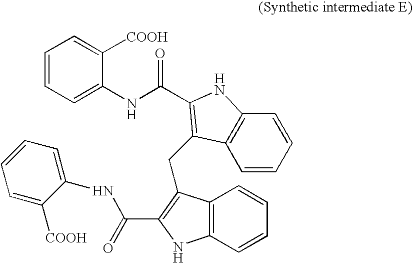

50 ml of acetic anhydride and 50 ml of toluene were added to 7.0 g of the synthetic intermediate E. The mixture was allowed to react under reflux for ten hours. After cooled with water, the precipitate was filtered and washed with water, to give 6.1 g of an exemplary compound (B-58) (yield: 93%).

MS: m/z 537 (M+)

¹H NMR (deuterated DMSO): δ 5.50 (2H), δ 6.79-6.83 (2H), δ 7.13-7.17 (2H), δ 7.34-7.36 (2H), δ 7.44-7.46 (2H), δ 7.56-7.60 (2H), δ 7.63-7.65 (2H), δ 7.89-7.93 (2H), δ 8.13-8.15 (2H), δ 11.82 (2H)

λmax=355 nm (EtOAc)

### Example 2-2

### Preparation of Exemplary Compound (B-75)

20 g of indole-2-carboxylic acid was added to a mixture of 27.8 g of potassium hydroxide and 200 ml of DMSO. The mixture was stirred at room temperature for one hour. After cooled to room temperature, 13.4 g of 1,4-dibromobutane was added to the mixture and stirred at room temperature for ten hours. Diluted hydrochloric acid was added dropwise to the mixture, and the solid obtained was filtered and washed with water, to give 21.9 g of a synthetic intermediate F (yield: 94%).

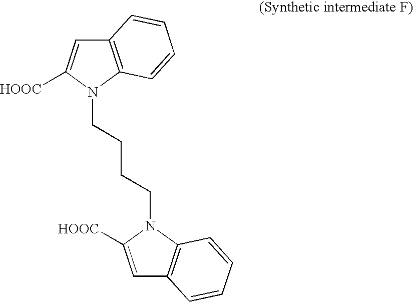

5.1 g of oxalyl chloride was added to a mixture of 5.0 g the synthetic intermediate F in 30 ml of toluene. After addition of a few drops of DMF, the mixture was stirred at room temperature for 30 minutes and at 80° C. for 10 minutes. The reaction solution was added to a solution of 3.6 g of anthranilic acid in 20 ml of dimethylacetamide, and the solid obtained was filtered and washed with water, to give 8.1 g of a synthetic intermediate G (yield: 99%).

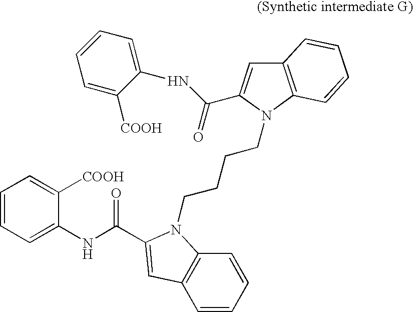

30 ml of acetic anhydride and 30 ml of toluene were added to 5.0 g of the synthetic intermediate G. The mixture was allowed to react under reflux for ten hours. After cooled with water, the precipitate was filtered and washed with water, to give 4.5 g of an exemplary compound (B-75) (yield: 96%).

MS: m/z 579 (M+)

¹H NMR (CDCl₃): δ 2.09 (4H), δ 4.87 (4H), δ 7.14-7.17 (2H), δ 7.32-7.45 (8H), δ 7.58 (2H), δ 7.61-7.65 (2H), δ 7.69-7.71 (2H), δ 8.14-8.16 (2H)

λmax=349 nm (EtOAc)

### <Preparation and Evaluation of Sample Solution>

One mg of exemplary compound (B-58) was dissolved in 100 ml of ethyl acetate, to give a sample solution. Similarly, sample solutions of exemplary compounds (B-75) and comparative compounds A and B respectively were prepared. The absorbance of each sample solution was determined in a 1 cm quartz cell by using Spectrophotometer UV-3600 (product name) manufactured by Shimadzu Corporation. The cell containing the sample solution was photoirradiated by a xenon lamp with its UV filter removed at an illuminance of 170,000 lux, and the amount of each compound remaining after irradiation for two days was determined. The residual amount was calculated according to the following Formula:

Residual amount (%)=100×(100−Transmittance after irradiation)/(100−Transmittance before irradiation)

The transmittance is a value determined at the maximum absorption wavelength of each compound. The result is shown in Table 17.

TABLE 17

| Sample | | Residual | |
|---|---|---|---|
| No. | Ultraviolet absorbent | amount (%) | |
| 1 | Exemplified | 95 | Present |
| | compound (B-58) | | invention |
| 2 | Exemplified | 94 | Present |
| | compound (B-75) | | invention |
| 3 | Comparative | 80 | Comparative |
| | compound A | | example |
| 4 | Comparative | 27 | Comparative |
| | compound B | | example |

As apparent from the results in Table 17, the ultraviolet absorbents according to the present invention remained in the sample solution in an amount greater than the comparative compounds A and B (conventional ultraviolet absorbents absorbing light in the UV-A range), indicating that these compounds were more resistant to decomposition by photoirradiation.

Having described our invention as related to the present embodiments, it is our intention that the invention not be limited by any of the details of the description, unless otherwise specified, but rather be construed broadly within its spirit and scope as set out in the accompanying claims.

This nonprovisional application claims priority under 35 U.S.C. §119 (a) on Patent Application No. 2007-95446 filed in Japan on Mar. 30, 2007, which is herein incorporated by reference.

1. An ultraviolet absorbent represented by the following Formula (1): 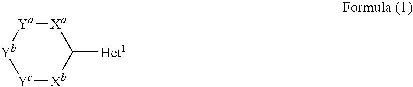
wherein, Het¹ represents a monovalent five- or six-membered aromatic heterocyclic residue; the aromatic heterocyclic residue may further be substituted;
X^{a} and X^{b} each independently represent a heteroatom, X^{a} and X^{b} may further be substituted;
Y^{a}, Y^{b} and Y^{c} each independently represent a heteroatom or a carbon atom; Y^{a}, Y^{b} and Y^{c} may further be substituted;
the ring formed from carbon atom, X^{a}, X^{b}, Y^{a}, Y^{b} and Y^{c} may have a double bond at any position;
the ring formed from carbon atom, X^{a}, X^{b}, Y^{a}, Y^{b} and Y^{c} has at least one fused ring.

2. The ultraviolet absorbent according to claim 1, wherein the ultraviolet absorbent represented by Formula (1) above is an ultraviolet absorbent represented by the following Formula (2): 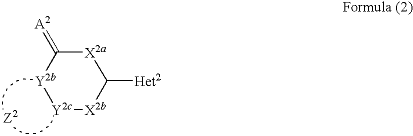
wherein, Het² is the same as Het¹ in Formula (1) above;
X^{2a} and X^{2b} each are the same as X^{a} and X^{b} in Formula (1) above;
Y^{2b} and Y^{2c} each are the same as Y^{b} and Y^{c} in Formula (1) above;
A² represents an oxygen atom or sulfur atom or ═NR^{a}, where R^{a} represents a hydrogen atom or a monovalent substituent group;
Z² represents an atom group needed for forming a four- to eight-membered ring together with Y^{2b} and Y^{2c}.

3. The ultraviolet absorbent according to claim 2, wherein the ultraviolet absorbent represented by Formula (2) above is an ultraviolet absorbent represented by the following Formula (3): 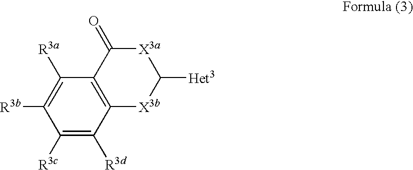
wherein, Het³ is the same as Het² in Formula (2) above;
X^{3a} and X^{3b} each are the same as X^{2a} and X^{2b} in Formula (2) above;
R^{3a}, R^{3b}, R^{3c} and R^{3d} each independently represent a hydrogen atom or a monovalent substituent group.

4. The ultraviolet absorbent according to claim 3, wherein the ultraviolet absorbent represented by Formula (3) above is an ultraviolet absorbent represented by the following Formula (4): 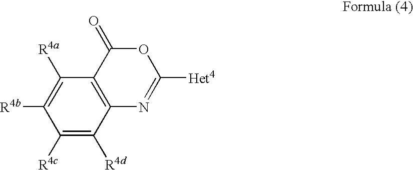
wherein, Het⁴ is the same as Het³ in Formula (3) above;
R^{4a}, R^{4b}, R^{4c} and R^{4d} are the same as R^{3a}, R^{3b}, R^{3c} and R^{3d} in Formula (3) above.

5. The ultraviolet absorbent according to claim 4, wherein the ultraviolet absorbent represented by Formula (4) above is an ultraviolet absorbent represented by the following Formula (5): 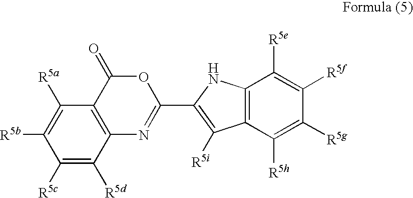
wherein, R^{5a}, R^{5b}, R^{5c} and R^{5d} are the same as R^{4a}, R^{4b}, R^{4c} and R^{4d} in Formula (4) above; R^{5e}, R^{5f}, R^{5g}, R^{5h} and R⁵ⁱ each independently represent a hydrogen atom or a monovalent substituent group.

6. An ultraviolet absorbent represented by the following Formula (6): 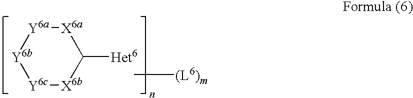
wherein, Het⁶ represents a monovalent five- or six-membered aromatic heterocyclic residue; the aromatic heterocyclic residue may further be substituted;
X^{6a} and X^{6b} each independently represent a heteroatom, X^{6a} and X^{6b} may further be substituted;
Y^{6a}, Y^{6b} and Y^{6c} each independently represent a heteroatom or a carbon atom; Y^{6a}, Y^{6b} and Y^{6c} may further be substituted;
L⁶ represents a bi- to octa-valent connecting group;
n is an integer of 2 or more, and m is an integer of 0 or more.

7. The ultraviolet absorbent according to claim 6, wherein the ring formed from carbon atom, X^{6a}, X^{6b}, Y^{6a}, Y^{6b} and Y^{6c} has at least one fused ring.

8. The ultraviolet absorbent according to claim 6, wherein the ultraviolet absorbent represented by Formula (6) above is an ultraviolet absorbent represented by the following Formula (7): 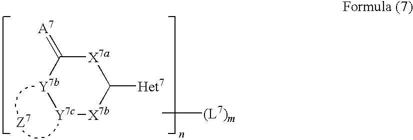
wherein, Het⁷ is the same as Het⁶ in Formula (6) above;
X^{7a} and X^{7b} each are the same as X^{6a} and X^{6b} in Formula (6) above;
Y^{7b} and Y^{7c} each are the same as Y^{6b} and Y^{6c} in Formula (6) above;
A⁷ represents an oxygen atom or sulfur atom or ═NR^{b}, where R^{b} represents a hydrogen atom or a monovalent substituent group;
Z⁷ represents an atom group needed for forming a four- to eight-membered ring together with Y^{7b} and Y^{7c};
L⁷ is the same as L⁶ in Formula (6) above;
n and m each are the same as n and m in Formula (6) above.

9. The ultraviolet absorbent according to claim 8, wherein the ultraviolet absorbent represented by Formula (7) above is an ultraviolet absorbent represented by the following Formula (8): 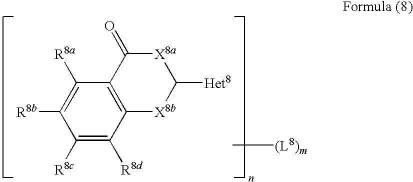
wherein, Het⁸ is the same as Het⁷ in Formula (7) above;
X^{8a} and X^{8b} each are the same as X^{7a} and X^{7b} in Formula (7) above;
R^{8a}, R^{8b}, R^{8c} and R^{8d} each independently represent a hydrogen atom or a monovalent substituent group;
L⁸ is the same as L⁷ in Formula (7) above;
n is an integer of 2 or more, and m is an integer of 0 or more.

10. The ultraviolet absorbent according to claim 9, wherein the compound represented by Formula (8) above is an ultraviolet absorbent represented by the following Formula (9): 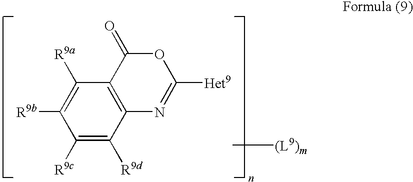
wherein, Het⁹ is the same as Het⁸ in Formula (8) above;
R^{9a}, R^{9b}, R^{9c} and R^{9d} are the same as R^{8a}, R^{8b}, R^{8c} and R^{8d} in Formula (8) above;
L⁹ is the same as L⁸ in Formula (8) above;
n is an integer of 2 or more, and m is an integer of 0 or more.

11. The ultraviolet absorbent according to claim 10, wherein the compound represented by Formula (9) above is an ultraviolet absorbent represented by the following Formula (10): 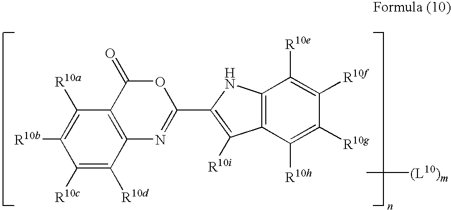
wherein, R^{10a}, R^{10b}, R^{10c} and R^{10d} are the same as R^{9a}, R^{9b}, R^{9c} and R^{9d} in Formula (9) above; R^{10a}, R^{10f}, R^{10g}, R^{10h} and R¹⁰ⁱ each independently represent a hydrogen atom or a monovalent substituent group;
L¹⁰ is the same as L⁹ in Formula (9) above;
n is an integer of 2 or more, and m is an integer of 0 or more.

12. A polymer material, comprising a polymer substance and the ultraviolet absorbent according to claim 1.

13. A polymer material, comprising a polymer substance and the ultraviolet absorbent according to claim 6.

14. A compound represented by the following Formula (6): 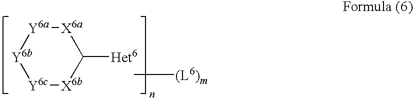
wherein, Het⁶ represents a monovalent five- or six-membered aromatic heterocyclic residue; the aromatic heterocyclic residue may further be substituted;
X^{6a} and X^{6b} each independently represent a heteroatom, X^{6a} and X^{6b} may further be substituted;
Y^{6a}, Y^{6b} and Y^{6c} each independently represent a heteroatom or a carbon atom;
Y^{6a}, Y^{6b} and Y^{6c} may further be substituted;
L⁶ represents a bi- to octa-valent connecting group;
n is an integer of 2 or more, and m is an integer of 0 or more.

15. The compound according to claim 14, wherein the compound is produced by using the compound represented by the following Formula (1): 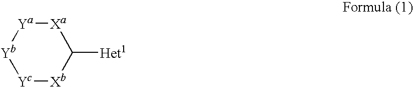
wherein, Het¹ is the same as Het⁶ in Formula (6) above;
X^{a} and X^{b} each are the same as X^{6a} and X^{6b} in Formula (6) above;
Y^{a}, Y^{b} and Y^{c} each are the same as Y^{6a}, Y^{6b} and Y^{6c} in Formula (6) above;
the ring formed from carbon atom, X^{a}, X^{b}, Y^{a}, Y^{b} and Y^{c} may have a double bond at any position;
the ring formed from carbon atom, X^{a}, X^{b}, Y^{a}, Y^{b} and Y^{c} has at least one fused ring.

16. The ultraviolet absorbent according to claim 7, wherein the ultraviolet absorbent represented by Formula (6) above is an ultraviolet absorbent represented by the following Formula (7): 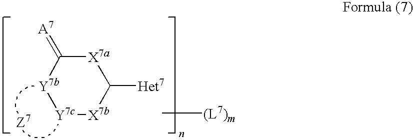
wherein, Het⁷ is the same as Het⁶ in Formula (6) above;
X^{7a} and X^{7b} each are the same as X^{6a} and X^{6b} in Formula (6) above;
Y^{7b} and Y^{7c} each are the same as Y^{6b} and Y^{6c} in Formula (6) above;
A⁷ represents an oxygen atom or sulfur atom or ═NR^{b}, where R^{b} represents a hydrogen atom or a monovalent substituent group;
Z⁷ represents an atom group needed for forming a four- to eight-membered ring together with Y^{7b} and Y^{7c};
L⁷ is the same as L⁶ in Formula (6) above;
n and m each are the same as n and m in Formula (6) above.

17. The ultraviolet absorbent according to claim 16, wherein the ultraviolet absorbent represented by Formula (7) above is an ultraviolet absorbent represented by the following Formula (8): 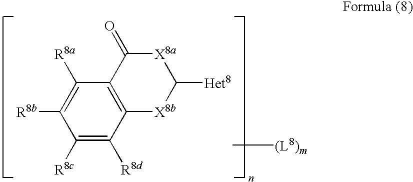
wherein, Het⁸ is the same as Het⁷ in Formula (7) above;
X^{8a} and X^{8b} each are the same as X^{7a} and X^{7b} in Formula (7) above;
R^{8a}, R^{8b}, R^{8c} and R^{8d} each independently represent a hydrogen atom or a monovalent substituent group;
L⁸ is the same as L⁷ in Formula (7) above;
n is an integer of 2 or more, and m is an integer of 0 or more.

18. The ultraviolet absorbent according to claim 17, wherein the compound represented by Formula (8) above is an ultraviolet absorbent represented by the following Formula (9): 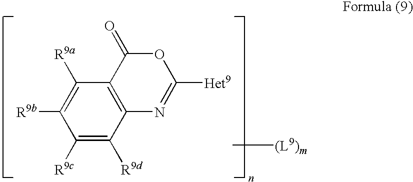
wherein, Het⁹ is the same as Het⁸ in Formula (8) above;
R^{9a}, R^{9b}, R^{9c} and R^{9d} are the same as R^{8a}, R^{8b}, R^{8c} and R^{8d} in Formula (8) above;
L⁹ is the same as L⁸ in Formula (8) above;
n is an integer of 2 or more, and m is an integer of 0 or more.

19. The ultraviolet absorbent according to claim 18, wherein the compound represented by Formula (9) above is an ultraviolet absorbent represented by the following Formula (10): 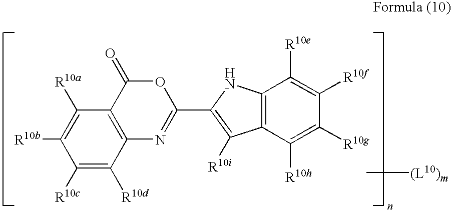
wherein, R^{10a}, R^{10b}, R^{10c} and R^{10d} are the same as R^{9a}, R^{9b}, R^{9c} and R^{9d} in Formula (9) above; R^{10e}, R^{10f}, R^{10g}, R^{10h} and R¹⁰ⁱ each independently represent a hydrogen atom or a monovalent substituent group;
L¹⁰ is the same as L⁹ in Formula (9) above;
n is an integer of 2 or more, and m is an integer of 0 or more.
